(19) United States
(12) Patent Application Publication
(10) Pub. No.: US 2025/0387404 A1
(43) Pub. Date: Dec. 25, 2025

(54) FORMULATIONS AND METHODS FOR TREATING EPIDERMOLYSIS BULLOSA SIMPLEX AND RELATED CONDITIONS

(71) Applicants: RUTGERS, THE STATE UNIVERSITY OF NEW JERSEY, New Brunswick, NJ (US); LEIPZIG UNIVERSITY, Leipzig (DE)

(72) Inventors: Bishr OMARY, New Brunswick, NJ (US); Thomas M. MAGIN, New Brunswick, NJ (US); Jacques Y. ROBERGE, New Brunswick, NJ (US); Pei LI, New Brunswick, NJ (US); Katrin RIETSCHER, New Brunswick, NJ (US)

(21) Appl. No.: 18/858,956

(22) PCT Filed: Apr. 24, 2023
(86) PCT No.: PCT/US2023/019611

§ 371 Date: Oct. 22, 2024

Related U.S. Application Data
(60) Provisional application No. 63/333,884, filed on Apr. 22, 2022.
(60) Provisional application No. 63/341,767, filed on May 13, 2022.

(51) Int. Cl. A61K 31/553; A61K 31/437; A61K 31/519; A61P 17/02

(52) U.S. Cl. CPC A61K 31/553; A61K 31/437; A61K 31/519; A61P 17/02

(57) ABSTRACT

The invention provides the use of kinase inhibitors and derivatives of staurosporine, such as PKC412, to treat patients having epidermis bullosa simplex (BBS), epidermolytic hyperkeratosis, epidermolytic palmoplantar keratoderma, palmoplantar keratoderma, nonepidermolytic, pachyonychia congenita type 1, pachyonychia congenita type 2, or other intermediate filament associated diseases using oral or topical cream or ointment formulations of the same. The invention also provides the use of SRC and RAF kinase inhibitors, alone or in combination with PKC412, to treat intermediate filament associated diseases.

### CROSS-REFERENCE TO RELATED APPLICATIONS

This patent application claims the benefit of priority of U.S. Provisional Patent Application No. 63/333,884, filed Apr. 22, 2022, and of U.S. Provisional Patent Application No. 63/341,767, filed May 13, 2022, which applications are herein incorporated by reference.

### STATEMENT OF GOVERNMENT SUPPORT

This invention was made with government support under DK47918 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Jul. 9, 2025, is named 08035_125US1_SL.xml and is 11,118 bytes in size.

### BACKGROUND

Mutations in intermediate filament proteins (IF) lead or predispose to more than 70 human diseases. Keratins make up the largest subgroup of IF proteins. Among IF-associated diseases, epidermis bullosa simplex (EBS) is a condition of the skin caused primarily by monoallelic amino acid substitutions in keratin 5 (K5) or keratin 14 (K14). Autosomal dominant mutations in KRT5 and KRT14, and in rare cases autosomal recessive inheritance, lead to EBS. Among all EB types, EBS is the most frequent, with approximately 1 case per 20,000 live births. In EBS, basal keratinocytes become fragile and show skin blistering upon mild trauma. Fluid-filled blisters and erosions occur in response to minor pressure or friction such as scratching. EBS symptoms vary widely among affected individuals. In mild cases, blistering primarily affects the hands and feet, and erosions usually heal without leaving scars. In severe EBS (EBS-S, also termed Dowling-Meara subtype; OMIM 131760) widespread blistering leads to large, eroded areas, pronounced inflammatory reactions, itch and potentially life-threatening complications (e.g., infection, sepsis, and even lethality) particularly during childhood. There is no known cure for EBS, and disease management involves supportive care to protect the skin from painful blistering and symptomatic treatment of blisters and erosions. Therefore, effective treatments for EBS are needed.

### BRIEF SUMMARY

As described herein, PP2 (1-tert-butyl-3-(4-chlorophenyl)-1H-pyrazolo[3,4-d]pyrimidin-4-amine), a SRC tyrosine kinase inhibitor, was discovered to be a new compound that normalizes K18 R90C-induced keratin filament aggregation and susceptibility to liver injury in experimental animals. The protective effect of PP2, as contrasted with PKC412, was found in male but not female mice, which is believed to be due to mouse sex differences in PP2 metabolism and occurs by serine and not tyrosine dephosphorylation of K8 and K18 without a change in NMHC-IIA phosphorylation (the latter, by contrast, is involved in the mechanism of action of PKC412). Knockdown of SRC, but not another kinase target of PP2, protein tyrosine kinase 6 (PTK6), blocked the protective effect of PP2. These findings lend support for the potential therapeutic use of kinase inhibitors in IF-associated diseases and show that inhibition of different kinase pathways provides a viable treatment or disease prevention approach.

In certain embodiments, provided here is a method for treating a subject having or at risk for developing an intermediate filament associated disease, comprising administering to the subject an effective amount of a SRC and/or RAF kinase inhibitor to treat an intermediate filament associated disease.

In certain embodiments, the subject is a human male.

In certain embodiments, the subject is a human female.

In certain embodiments, the kinase inhibitor is PP2, or a salt thereof, or vemurafenib, or a salt thereof.

In certain embodiments, the intermediate filament associated disease is epidermis bullosa simplex, epidermolytic hyperkeratosis, epidermolytic palmoplantar keratoderma, palmoplantar keratoderma, nonepidermolytic, pachyonychia congenita type 1, or pachyonychia congenita type 2.

Other objects, features, and advantages of the present invention will be apparent to one of skill in the art from the following detailed description and figures.

Epidermolysis bullosa simplex (EBS) is a severe and potentially life-threatening disorder for which no adequate therapy exists. Most cases are caused by dominant mutations in keratins KRT5 or KRT14, leading to the formation of cytoplasmic keratin aggregates, profound keratinocyte fragility and cytolysis. As described herein, it was hypothesized that pharmacological reduction of keratin aggregates, which compromises keratinocyte cell integrity and leads to cell death upon exposure to limited environmental stresses that normally would otherwise be well tolerated, represents a viable strategy for the treatment of EBS. Herein it is shown that the multi-kinase inhibitor PKC412, which is currently in clinical use for acute myeloid leukemia and advanced systemic mastocytosis, reduced keratin aggregation by 40% in patient-derived K14.R125C EBS-associated keratinocytes. Using a combination of epithelial shear stress assay and real-time impedance spectroscopy, it is shown that PKC412 restored intercellular adhesion. Molecularly, global phosphoproteomic analysis together with immunoblots, using phospho-epitope specific antibodies, revealed that PKC412 treatment altered phospho-sites on keratins and desmoplakin. Thus, provided herein is proof of concept for repurposing existing drugs for the targeted treatment of EBS and related conditions. Also described is how one broad-range kinase inhibitor reduced keratin filament aggregation in patient-derived EBS keratinocytes and fragility of EBS cell monolayers. These discoveries pave the way for a clinical trial using PKC412 for systemic or local application of patients with EBS or related conditions.

Accordingly, certain embodiments also provide a method to treat a human patient having epidermis bullosa simplex (EBS), epidermolytic hyperkeratosis, epidermolytic palmoplantar keratoderma, palmoplantar keratoderma, nonepidermolytic, pachyonychia congenita type 1, or pachyonychia congenita type 2, comprising administering to the patient a therapeutically effective amount of the compound PKC412 having the following structure I

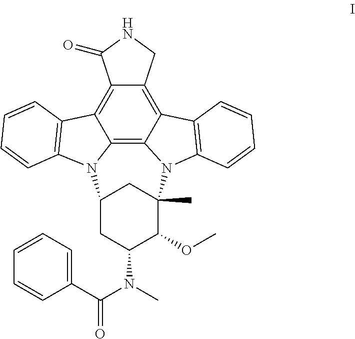 [SMILES: CO[C@@H]1[C@H](N(C)C(=O)c2ccccc2)C[C@H]2C[C@]1(C)n1c3ccccc3c3c4c(c5c6ccccc6n2c5c31)C(=O)NC4]

or a salt thereof.

In certain embodiments, the PKC412, or salt thereof, is administered orally.

In certain embodiments, the PKC412, or salt thereof, is administered topically to the patient's skin.

In certain embodiments, the PKC412, or salt thereof, is administered topically to the patient's skin, e.g., in a cream or ointment formulation.

In certain embodiments, the formulation for topical administration, e.g., the cream or ointment formulation, comprises a concentration of about 0.1% to 5% PKC412, or a salt thereof.

In certain embodiments, the formulation for topical administration, e.g., the cream or ointment formulation, comprises a concentration of about 0.5% PKC412, or a salt thereof.

Certain embodiments provide a formulation for topical administration, e.g., a cream or ointment formulation, that comprises a therapeutically effective amount of the compound PKC412 having the following structure I

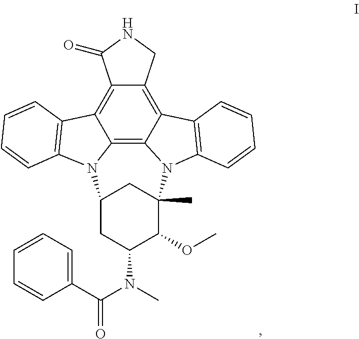 [SMILES: CO[C@@H]1[C@H](N(C)C(=O)c2ccccc2)C[C@H]2C[C@]1(C)n1c3ccccc3c3c4c(c5c6ccccc6n2c5c31)C(=O)NC4]

or a salt thereof.

In certain embodiments, the formulation comprises a concentration of about 0.1% to 5% PKC412, or a salt thereof.

In certain embodiments, the formulation comprises a concentration of about 0.5% PKC412, or a salt thereof.

Certain embodiments provide the use of the compound PKC412, or a salt thereof, to treat epidermis bullosa simplex (EBS), epidermolytic hyperkeratosis, epidermolytic palmoplantar keratoderma, palmoplantar keratoderma, nonepidermolytic, pachyonychia congenita type 1, or pachyonychia congenita type 2.

Certain embodiments provide the use of a formulation for topical administration, e.g., a cream or ointment formulation, that comprises the compound PKC412, or a salt thereof, to treat epidermis bullosa simplex (EBS), epidermolytic hyperkeratosis, epidermolytic palmoplantar keratoderma, palmoplantar keratoderma, nonepidermolytic, pachyonychia congenita type 1, or pachyonychia congenita type 2.

Certain embodiments provide the use of the compound PKC412, or a salt thereof, or the use of a cream or ointment formulation for topical administration that comprises the compound PKC412 or a salt thereof, to reduce keratin filament aggregation, e.g., in a patient in need thereof.

In certain embodiments, the PKC412, or a salt thereof, is in combination with a RAF kinase inhibitor, such as vemurafenib, or a salt thereof.

Other objects, features, and advantages of the present invention will be apparent to one of skill in the art from the following detailed description and figures.

### BRIEF DESCRIPTION OF THE FIGURES

FIG. 1. Mutation-specific keratin organization during steady state culture conditions and upon heat stress. (1a) The EBS-associated K5/K14 mutations used in this study are shown. (1b) Immunofluorescence analysis of immortalized patient-derived normal human keratinocytes (NHK) or K14.R125C cells. Maximum intensity projections of at least 15 optical sections are depicted. K5 staining revealed an intact keratin cytoskeleton of normal human keratinocytes (NHK) cell line, whereas K14.R125C keratinocytes showed keratin aggregates at the cell periphery under normal culture conditions (37° C.). Note the modest filament bundling in NHK upon elevated temperature (42° C., 15 min), but the extensive increase in keratin aggregation in K14.R125C cells, followed by a decrease during recovery (37° C., 1-3 h). Each inset is enlarged in the ‘detail’ panels. Scale bar=50 μm, detail=25 μm.

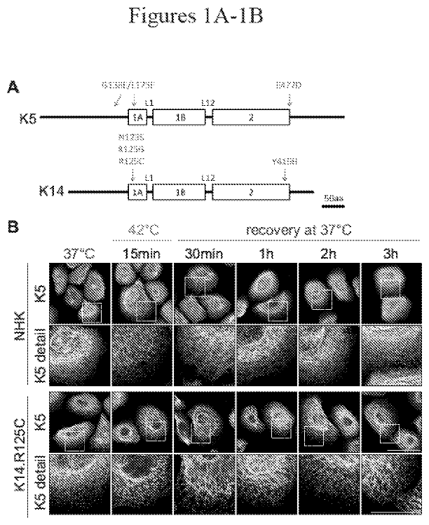

FIG. 2. PKC412 reduces keratin aggregation and improves epithelial sheet stability in K14.R125C keratinocytes. (2a) PKC412 reduced keratin aggregates in K14.R125C cells, depicted by K5 staining. Normal human keratinocytes (NHK) or K14.R125C cells were cultured with DMSO or PKC412 for 24 h and analyzed by immunofluorescence microscopy. Maximum intensity projections of confocal sections are depicted. Scale bar=50 μm, detail=10 μm. (2b) Graph depicting the significant changes of K14.R125C keratinocytes treated with PKC412 in comparison to DMSO-treated EBS cells (median is shown, 2-tailed Student's t-test, n=3 with >100 counted cells each experiment, ***P<0.001). (2c) Dispase assay of NHK and K14.R125C treated with DMSO or PKC412 in high calcium medium for 24 h and its quantification (2d) (median is shown, n=6, 2 way-ANOVA, Sidak's multiple comparisons test, *P<0.05, ***P<0.001).

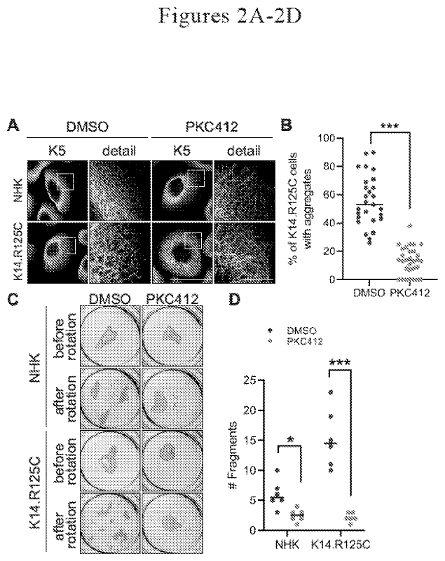

FIG. 3. The kinase inhibitor PKC412 increases relative impedance of K14.R125C keratinocytes. (3a) Assay principle of multielectrode array-based impedance spectroscopy. (3b) Impedimetric time course of electrodes covered by K14.R125C or NHK, cultured for two days in low calcium medium (LCM) followed by high calcium medium culture to form intercellular contacts (mean±SEM, n=3). (3c) Relative |Z|_{MAX} for EBS keratinocytes in comparison to NHK at depicted time points normalized to LCM values (mean±SEM, n=4, 2 way ANOVA, Sidak's multiple comparisons test; ***P<0.001). (3d) Relative |Z|_{MAX}/% for EBS keratinocytes in comparison to NHK treated with different PKC412 concentrations at 24 h after calcium switch (mean±SEM, n=4, 1 way ANOVA, Tukey's multiple comparisons test; ***P<0.001, n.s.=not significant). (3e) Impedance spectroscopy based EC₅₀ quantification for PKC412 treatment of K14.R125C keratinocytes and NHK (mean±SEM, n=4).

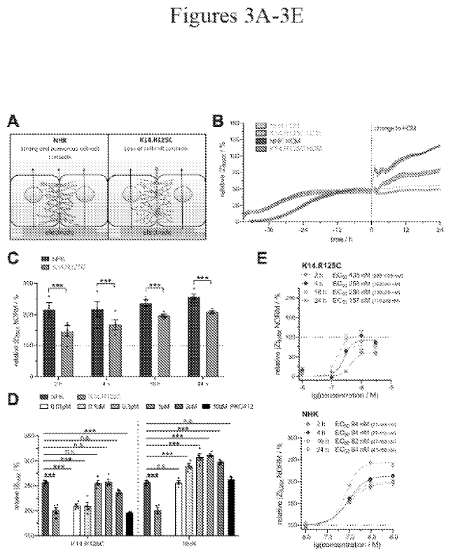

FIG. 4. PKC412 alters keratin phosphorylation. (4a) WB of PKC412- or DMSO-treated NHK and K14.R125C cells. A Coomassie gel shows equal protein loading. (4b) Graph depicting the relative RXXpS/pT signal from PKC412-treated NHK and K14.R125C keratinocytes normalized to DMSO-treated cells (mean±SD, n=3, 2 way-ANOVA, Sidak's multiple comparisons test, n.s.=not significant, **P<0.01). (4c) GOBP annotation enrichment analysis. Fisher exact test using Benj. Hoch. FDR truncation value of 0.01. (4d) WB of PKC412- or DMSO-treated EBS cells and NHK. (4e) The graph depicts the P-K17 S44 signal relative to total K17 from PKC412-treated NHK and K14.R125C cells normalized to DMSO-treated cells (mean±SD, n=4, 2 way-ANOVA, Sidak's multiple comparisons test, ***P<0.001).

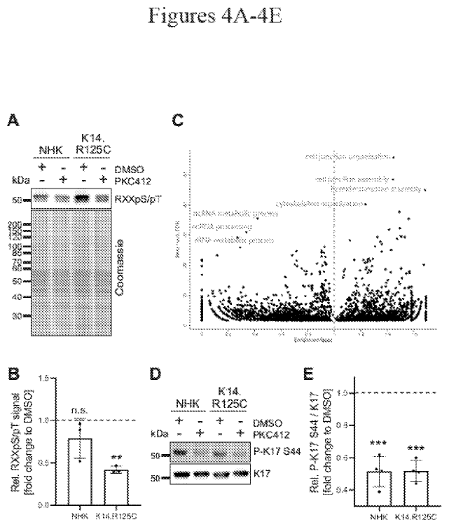

FIG. 5. PKC412 affects DSP phosphorylation and localization. (5a) WB of PKC412- or DMSO-treated EBS cells and NHK for total and P-DSP S165/S166 and (5b) its quantification (mean±SD, n=4, 2 way-ANOVA, Sidak's multiple comparisons test, ***P<0.001). Total DSP and α-tubulin blots show equal protein loading. (5c) Confocal immunofluorescence shows PKC412- or DMSO-treated NHK and K14.R125C keratinocytes stained with K5 (in red) and DSP (in green). Scale bar=50 μm, detail=10 μm. (5d) DSP fluorescence intensity along cell borders was quantified (>200 cell borders, n=3, 2 way ANOVA, Sidak's multiple comparisons test: ***P<0.001, n.s.=not significant). (5e) WB of PKC412- or DMSO-treated EBS cells and NHK for total and P-DSP S2849 and (5f) its quantification (mean±SD, n=4, 2 way-ANOVA, Sidak's multiple comparisons test, ***P<0.001). Total DSP and α-tubulin blots show equal protein loading.

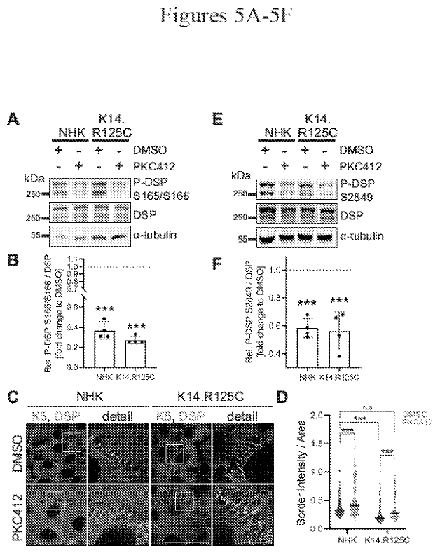

FIG. 6. Model depicting the changes in EBS K14.R125C keratinocytes upon PKC412 treatment. (6a) EBS K14.R125C keratinocytes show keratin aggregates localized preferentially at the cell periphery. Desmosomes are sparse and as a consequent intercellular cohesion of K14.R125C epithelial sheets is strongly decreased in comparison to normal human keratinocytes. Treatment of K14.R125C keratinocytes with the multi-kinase inhibitor PKC412 (6b) decreases keratin aggregation and enhances the localization of the desmosomal linker protein desmoplakin (DSP) at sites of cell-cell contacts leading to strengthened intercellular cohesion in K14.R125C keratinocytes. PKC412 directly affects keratin phosphorylation and indirectly keratin-associated proteins such as DSP. PKC412 treatment reduces phosphorylation of K17 at S44 as well as of DSP at S165, S166 and S2849.

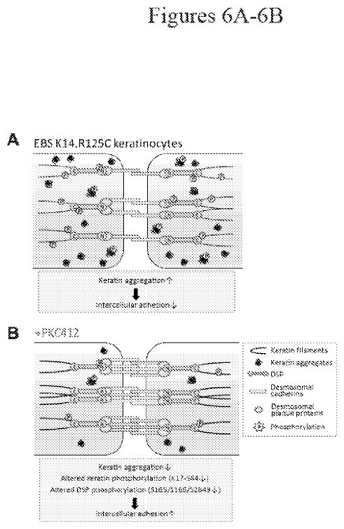

FIG. 7. Impact of distinct EBS mutations on keratin intermediate filament organization under steady state culture conditions, upon heat stress and recovery. Immortalized patient derived K14.R125G, K14.Y415H, K14.N123S, K5.G138E/L175F and K5.E477D keratinocytes were analyzed by immunofluorescence microscopy showing K5. Maximum intensity projections of at least 15 optical sections are depicted. Keratinocytes were cultured using normal culture conditions (37° C.), or at elevated temperature (heat stress, 42° C. for 15 min) then recovered (37° C., 1-3 h). Each inset is enlarged in the ‘detail’ panels. Scale bar=50 m, detail=25 μm.

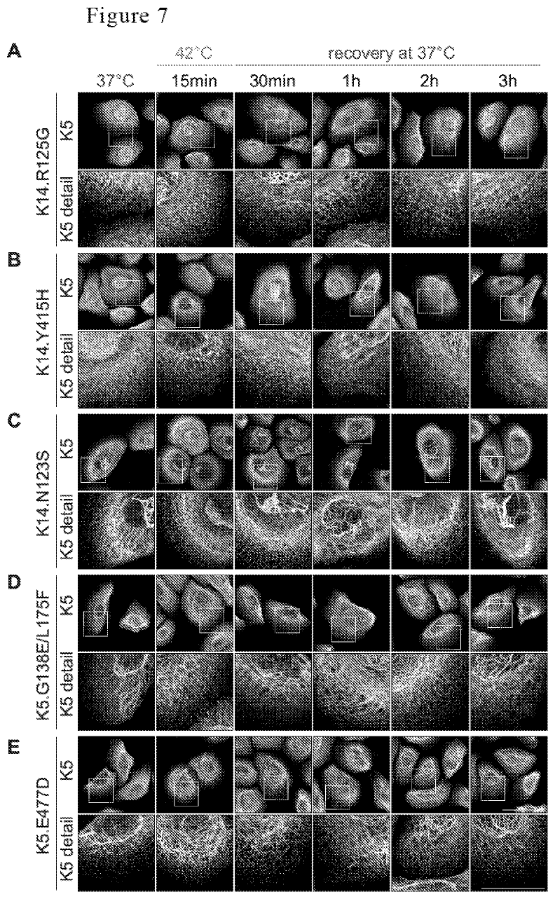

FIG. 8. PKC412 treatment improves epithelial sheet integrity of K14.R125C and K14.R125G keratinocytes. (A) Model of epithelial sheet assay. (B) PKC412 treatment improves epithelial sheets of K14.R125C and K14.R125G keratinocytes shown by dispase assay. Normal human keratinocytes (NHK) and K14.R125C/G cells were seeded and grown to confluency, cultured with DMSO or PKC412 in high calcium medium (HCM) for 24 h and treated with dispase. Freefloating cell monolayers were documented (before rotation), suspended to overhead rotation and documented (after rotation). For better comparison NHK and K14.R125C data were included. (C) Scatter blot depicting the number of cell fragments after rotation for NHK, K14.R125C and K14.R125G keratinocytes treated with DMSO or PKC412, respectively (median is shown, n=6 for NHK and K14.R125C, n=9 for K14.R125G, 2 way-ANOVA, Sidak's multiple comparisons test, *P<0.05, ***P<0.001). For better comparison NHK and K14.R125C data were included.

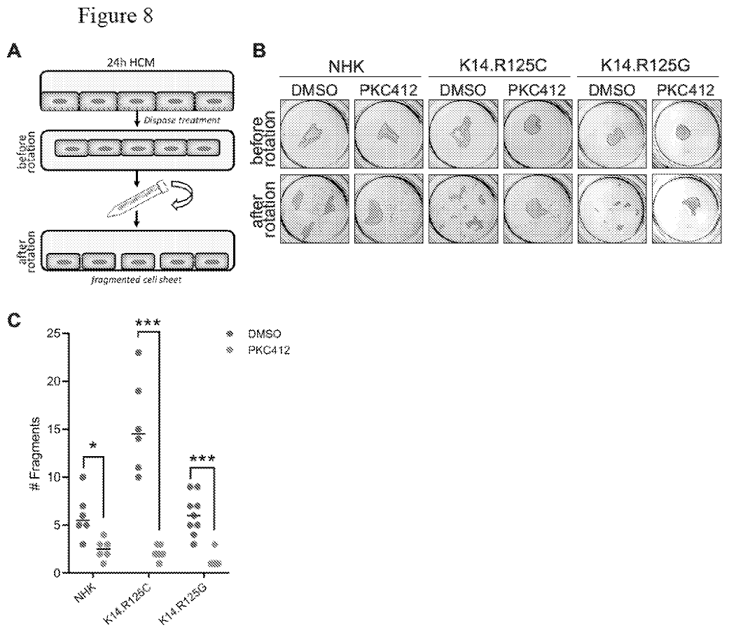

FIG. 9. Concentration- and time-dependent effect of PKC412. Graph depicting the significant changes of relative |Z|MAX/% for K14.R125C keratinocytes (A) and NHK (B) treated with different PKC412 concentrations at 2 h, 4 h, 16 h and 24 h after calcium switch normalized to NHK treated with DMSO (mean±SEM, n=4, 1 way ANOVA, Tukey's multiple comparisons test: *P<0.05, **P<0.01, ***P<0.001, n.s.=not significant).

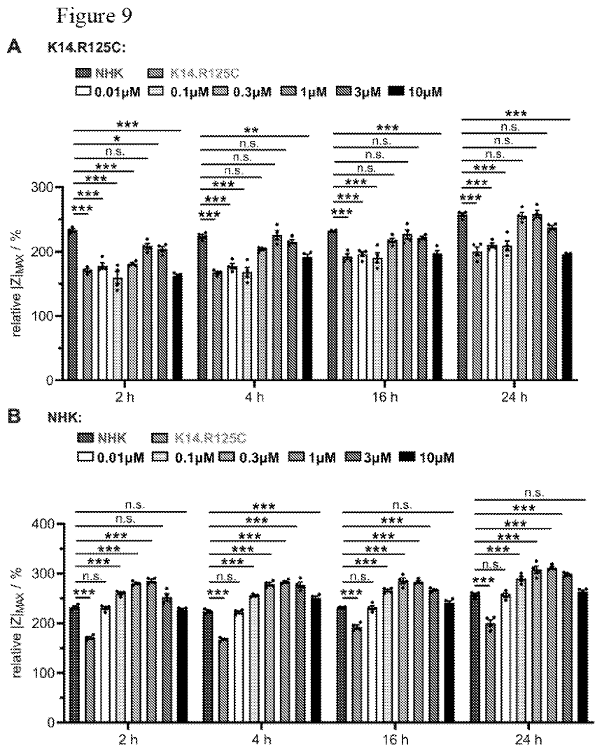

FIG. 10. The K14.R125C mutation is accompanied by altered phosphorylation. (A) MMIMP and MusiteDeep algorithm-based prediction of a loss of phosphorylation at K14.S128 (125CLASY) in the presence of K14.R125C mutation. Figure discloses SEQ ID NOS 1-2, respectively, in order of appearance. (B) The predicted kinases recognizing S128 are listed in order of decreasing probability (top to bottom). (C) Motif annotation enrichment analysis of phosphosites. Fisher exact test using Benj. Hoch. FDR truncation value of 0.01. The three most significantly downregulated motif categories are annotated.

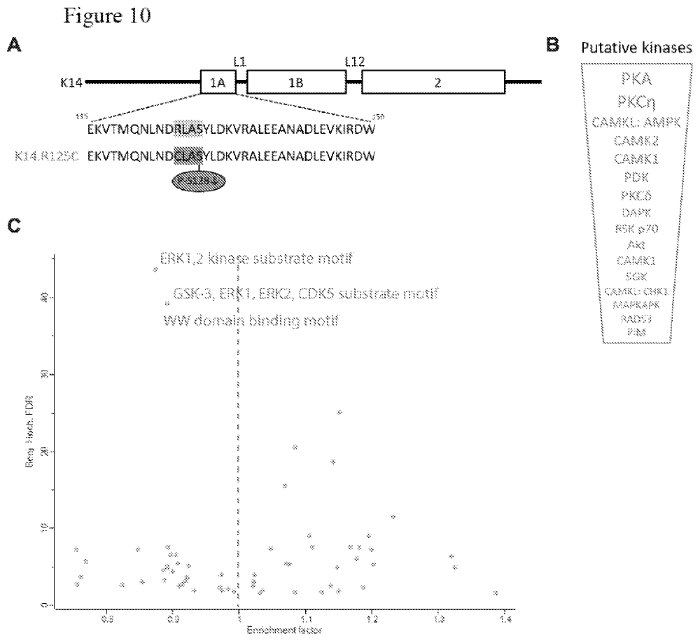

FIG. 11. PKC412 treatment alters keratin and desmoplakin phosphorylation. (A) Phospho-proteome analysis revealed decreased phospho-sites in K5, K14, K6A and K17 upon PKC412 treatment of K14.R125C keratinocytes in comparison to DMSO-treated cells. K6B and K16 showed no reduced phospho sites. Protein sequences for the keratins are shown. Decreased phospho sites upon PKC412 treatment are highlighted in red. The rod domain is shown in grey. Figure discloses SEQ ID NOS 3-8, respectively, in order of appearance. (B) The table shows all reduced serine (S), threonine (T) and tyrosine (Y) residues in K5, K14, K6A and K17, which were less phosphorylated upon PKC412 treatment in comparison to DMSO-treated K14.R125C keratinocytes. Asterisks denote amino acids covering the AGC kinase motif RXXpS. (C) Domain structure of desmoplakin (DSP) showing the central rod domain, the amino-terminal head domain (DSP-NT) and the carboxy-terminal tail domain (DSP-CT) with its three plakin repeat domains. Phospho-proteome analysis identified six phospho sites (P) within the desmosome binding domain (DSP-NT) and nine phospho sites within the intermediate filament (IF) binding domain (DSP-CT), that were reduced phosphorylated upon PKC412 treatment in comparison to DMSO-treated K14.R125C keratinocytes.

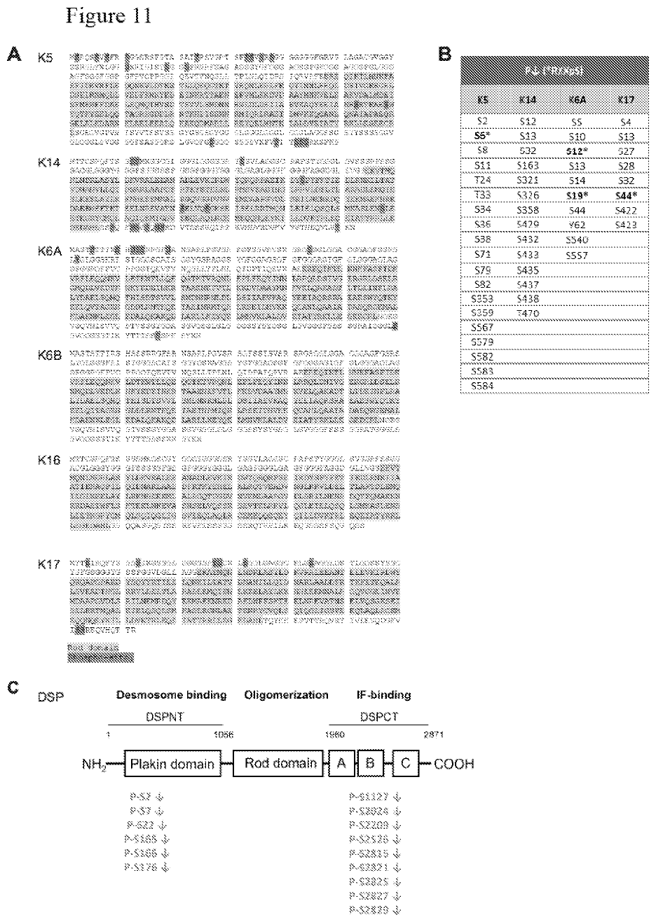

FIG. 12. PP2 corrects K18 R90C induced filament disruption and protects from Fas-induced apoptosis in cultured A549 cells. (A) Representative images and (B) quantification of GFP-K18 R90C lentivirus-transduced A549 cells treated with vehicle dimethyl sulfoxide (DMSO) or PP2 (0.5 μM/1 μM/5 μM) for 48 h. The average percent+SD of green fluorescent protein (GFP) K18-expressing cells with dots is measured. Representative “dots” and “filaments” are labeled with arrows and arrowheads, respectively. ***p<0.001 when comparing panels d with a. Scale bar=50 μm. For panel B, each circle represents an image field. N=5 with >200 cells/condition/experiment. (C) GFP-K18 wild-type (WT) or GFP-K18 R90C lentivirus-transduced A549 cells were treated with DMSO (−) or PP2 (+, 5 μM) for 48 h followed by treatment of IFN-γ (40 ng/mL, 6 h) then Fas ligand (FasL) (100 ng/mL, 12 h) to induce apoptosis. Cell lysates were analyzed by blotting using antibodies to the indicated apoptosis markers. Coomassie staining shows equal protein loading. The average relative intensity (±SD) of the indicated bands from 3 individual experiments is included below the blot. **p<0.01 when comparing K18 R90C-expressing cells with or without PP2. (D) Representative TUNEL staining of GFP-K18 R90C transduced A549 cells treated with DMSO or PP2 (5 μM) for 48 h then challenged with IFN-γ and FasL or PBS. Average percent+SD of TUNEL-positive cells (highlighted by arrows) is measured. ***p<0.001 when comparing the upper row panels. Scale bar=100 μm. N=3 with >1000 cells/condition/experiment.

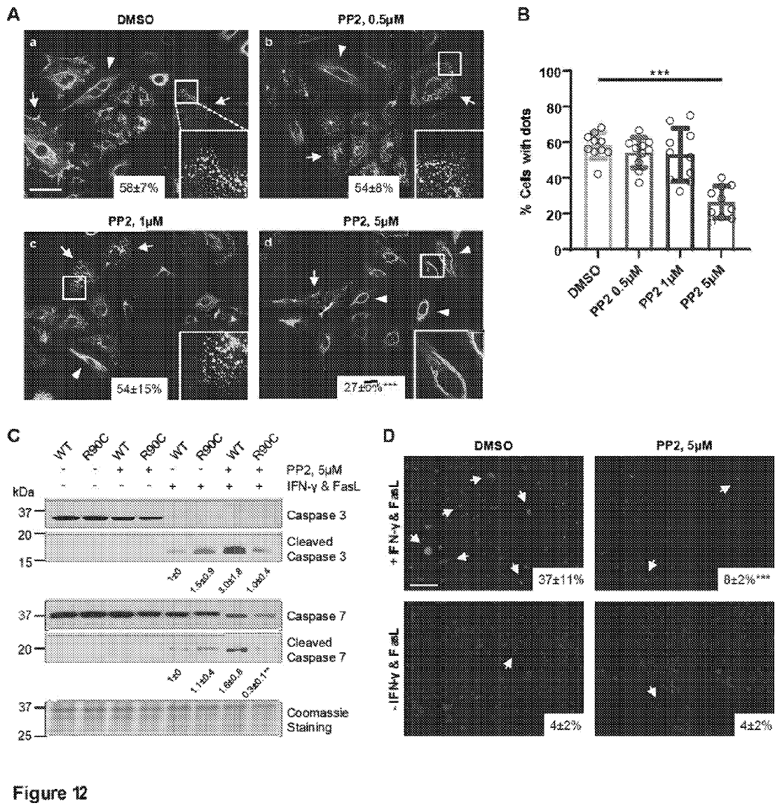

FIG. 13. PP2 treatment protects from Fas-induced liver injury in male but not female K18 R90C mice. K18 R90C mice were treated daily with DMSO or PP2 (1 mg/kg body weight; intraperitoneally) for 4d, then challenged with vehicle (PBS) or Fas antibody (0.15 μg/g body weight; 5 h) to induce liver injury. *p<0.05, ***p<0.001. (A) Liver sections were stained with anti-K18 antibody and analyzed by GFP fluorescence. Note the increase in number of cells with normalized keratin filament organization (panel d, arrow heads). The numbers show average percent±SD of cells with dots (arrows), which decreased significantly in male but not female livers after PP2 treatment. Scale bar=20 μm. N=2 with 4-6 mice per group. (B, C) Representative images and quantification of the hemorrhage levels observed in hematoxylin and eosin-stained liver sections. Scale bar=20 μm. (D) The increase in serum alanine aminotransferase (ALT) after Fas-L treatment becomes significantly reduced upon PP2 pretreatment in male but not female mice. (E) Mouse liver TUNEL (terminal deoxynucleotidyl transferase dUTP nick end labeling)-positive cells in livers of female and male mice relative to the −PP2/+Fas female group. For panels C-E, each circle or triangle symbol represents one mouse.

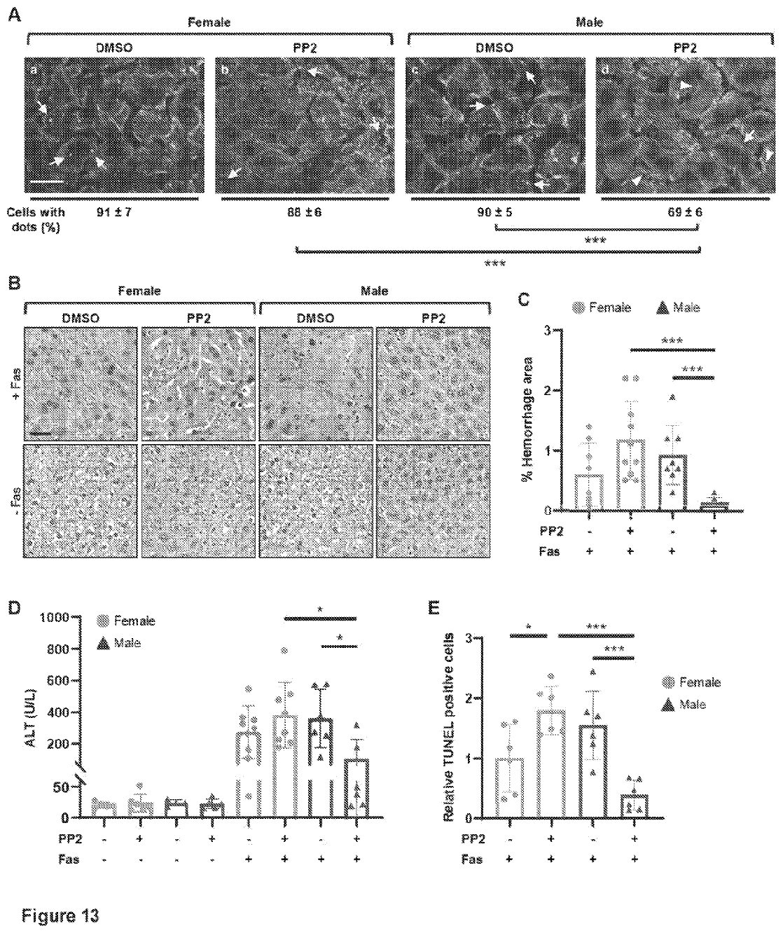

FIG. 14. PP2 blocks pro-apoptotic changes and SRC kinase activation in male but not female livers of K18 R90C mice upon Fas-induced apoptosis. K18 R90C mice were treated daily with DMSO or PP2 (1 mg/kg body weight; intraperitoneally) for 4d, then challenged with Fas antibody (0.15 μg/g body weight; 5 h). (A) Liver lysates (each lane represents independent livers) were immunoblotted with antibodies to the indicated antigens. Coomassie staining is included to show equal loading. (B) Relative intensities of the indicated protein bands (hK18=human K18) displayed in panel A were quantified. Each circle or triangle symbol represents one mouse. The relative SRC-pY416 levels were determined by dividing SRC-pY416 by total SRC band intensities. N=4 with 2-4 mice/group/experiment. *p<0.05, **p<0.01.

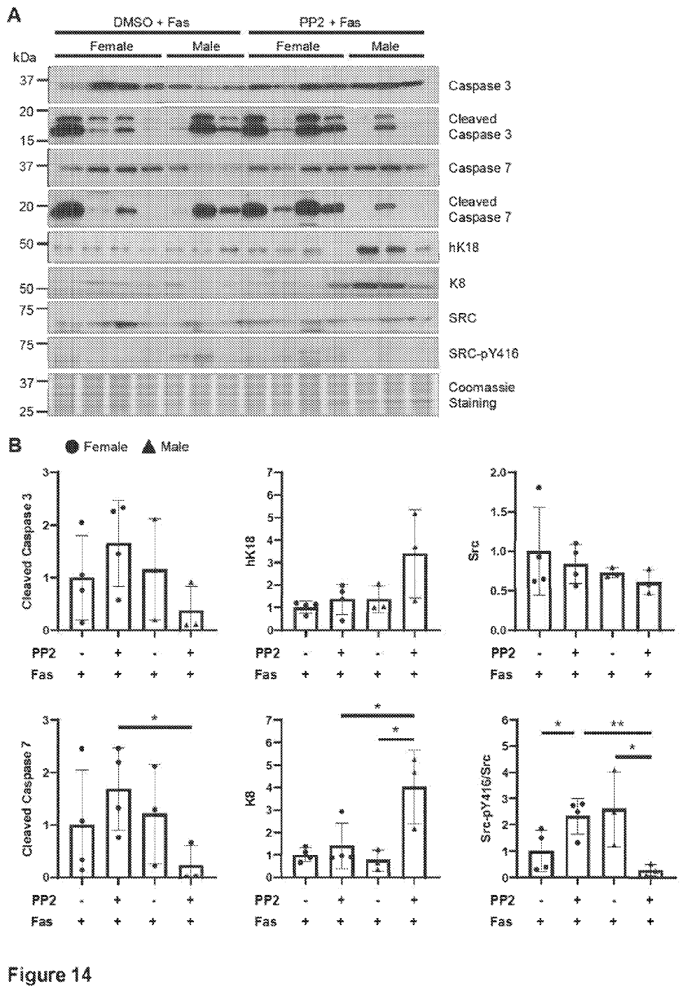

FIG. 15. SRC but not PTK6 expression is required for PP2-mediated keratin filament normalization. (A-C) GFP-K18 R90C A549 cells were cultured with scrambled or SRC siRNA (24 h) followed by treatment of DMSO or PP2 (5 μM; 48 h). (A) Immunoblot analyses using antibodies to SRC, K8/K18 and GFP. (B) Fluorescence microscopy and (C) quantification of the cells with dots based on keratin (green) and nuclear 4′,6-diamidino-2-phenylindole (DAPI) staining. The average percent±SD of GFP K18-expressing cells with dots is measured. ***p<0.001 when comparing panel b with a, and b with d (panel B). Scale bar=20 μm. (D-F) An identical experiment to that shown in panels A-C was carried out (including the analysis) except that GFP-K18 R90C A549 cells were cultured with scrambled or PTK6 siRNA (24 h) followed by the addition of DMSO or PP2 (5 μM; 48 h). ***p<0.001 when comparing panel b with a, and d with c (panel E). Scale bar=20 μm. For panels C and F, each circle represents a single image field. N=3 with >100 cells/condition/experiment.

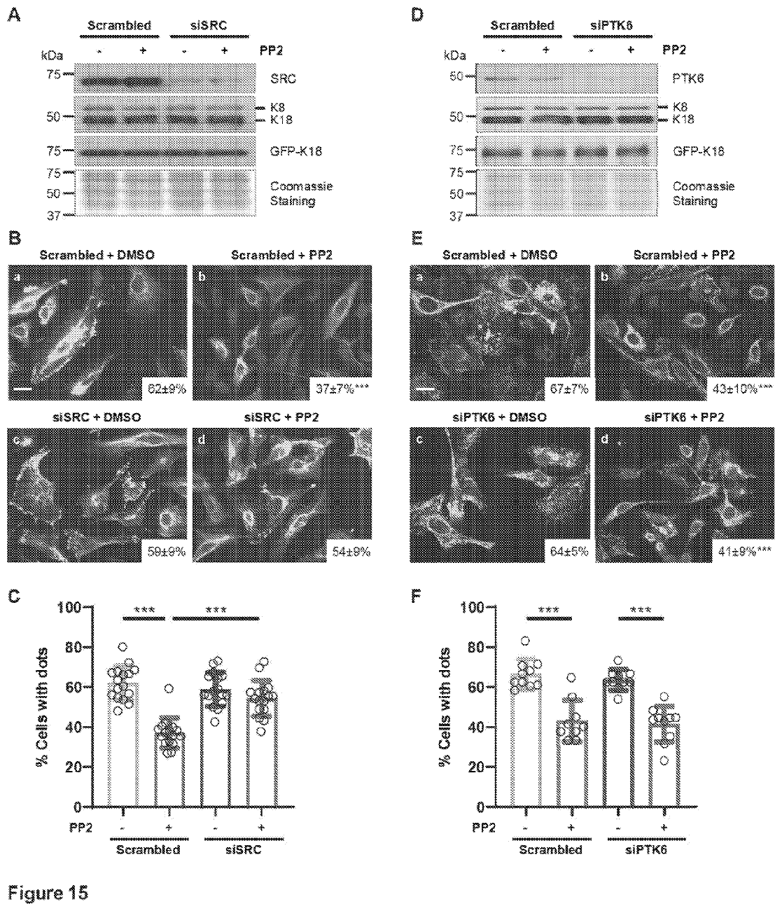

FIG. 16. PP2 decreases K18 and K8 phosphorylation in cultured A549 cells. (A) The effect of PP2 on K18 and K8 phosphorylation was analyzed by mass spectrometry. Peptides found in both DMSO and PP2 groups were filtered. PP2/DMSO represents the relative abundance ratio of the phospho-peptides normalized to the levels of K18 or K8. (B) GFP-K18 R90C transduced A549 cells were treated with DMSO, PP2 (5 μM) or PKC412 (0.8 μM) for 48 h. Cells were homogenized in 1% NP40-containing buffer followed by immunoprecipitation with anti-K8/K18 antibody. Lysates (input) or keratin immunoprecipitates were blotted with antibodies to the indicated antigens. PKC412 was used as ‘negative control’ kinase inhibitor since it has no effect on K18 S34 and K8 S432 phosphorylation based on prior studies (19). (C) Relative intensity of the indicated phospho-keratins was quantified (***p<0.001). Each circle represents an independent experiment.

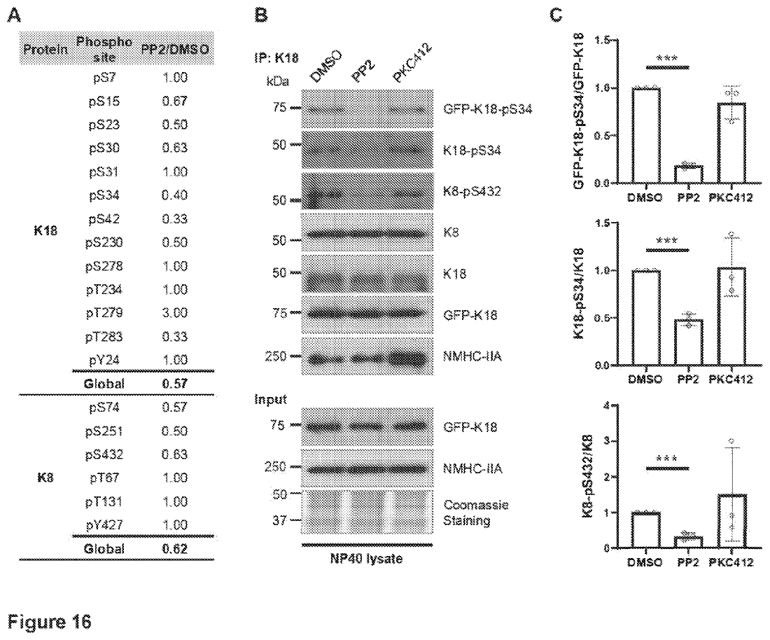

FIG. 17. Vemurafenib protects from Fas-induced apoptosis in cultured cells and male but note female K18 R90C mice. (A) GFP-K18 R90C transduced A549 cells were treated with vehicle (DMSO) or the RAF kinas inhibitor vemurafenib (1 μM/5 μM/20 μM) for 48 h followed by the addition of IFN-γ (40 ng/mL; 6 h) then FasL (100 ng/mL; 12 h) to induce apoptosis. Cell lysates were analyzed by immunoblotting using antibodies to the indicated antigens. The actin blot is included as a loading control. N=2. (B) Representative TUNEL staining of GFP-K18 R90C transduced A549 cells treated with DMSO or vemurafenib (5 μM) for 48 h then challenged with IFN-γ and FasL or PBS. Average percentage±SD of TUNEL-positive cells (highlighted by arrows) is measured. ***p<0.001 when comparing the upper row panels. Scale bar=50 μm. N=2 with >500 cells/condition/experiment. (C-F) K18 R90C mice were treated daily with DMSO (−) or vemurafenib (Vem) at 1 mg/kg (+) or 5 mg/kg (++) body weight intraperitoneally for 4d then challenged with Fas antibody (0.15 μg/g body weight; 5 h). Liver H&E staining (scale bar=20 μm) and hemorrhage levels, serum ALT and liver immunoblot test were used to assess liver injury. For panels D and E, each circle or triangle symbol represents one mouse. *p<0.05. For panel F, livers lysates (each lane represents independent livers) were immunoblotted with antibodies to the indicated antigens. Coomassie staining is included to show equal loading. N=2 with 2-4 mice/group/experiment.

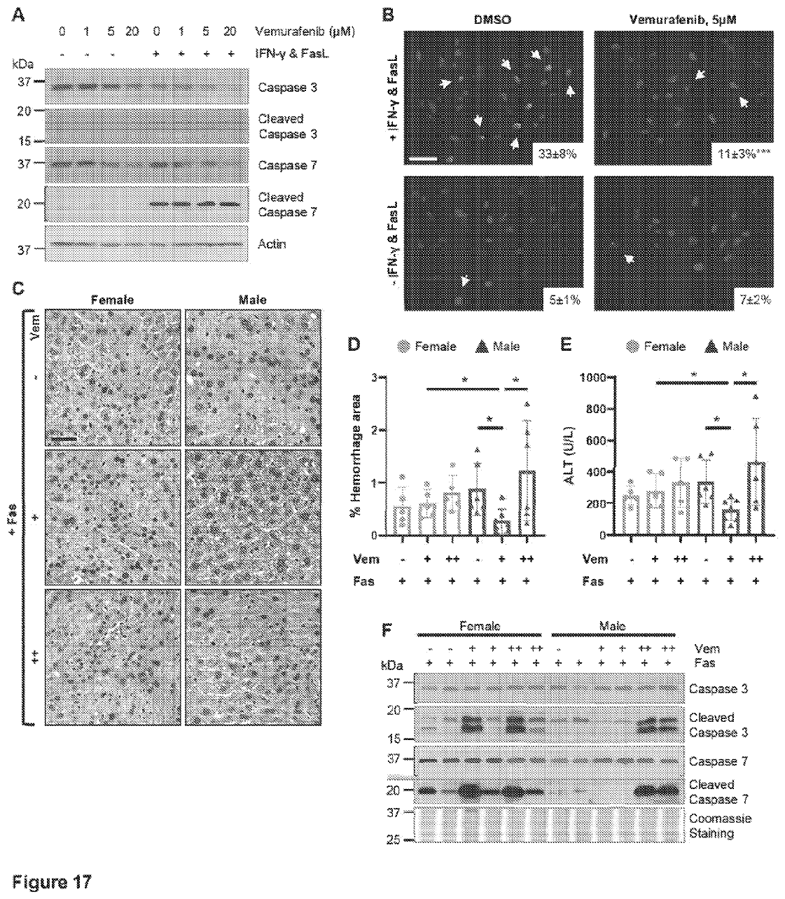

FIG. 18. Livers of male mice retain more PP2 than female mice. (A, B) Representative chromatograms (absorbance plotted against elution time) of male and female mouse livers and serum samples harvested at different time points after PP2 intraperitoneal injection of mice (overlayed with 100 picomole of PP2 standard). (C) UV-visible spectra of the eluting peaks shown in panel A. (D) Changes in relative PP2 levels (with respect to male mice liver at time 1 h) in male and female mouse liver as a function of time. Note the rapid turnover of PP2 in female as compared with male livers (**p<0.01).

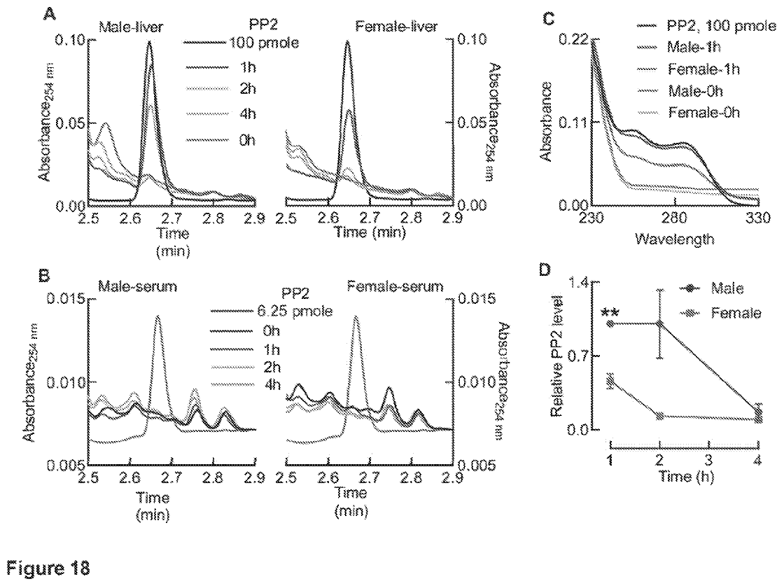

FIG. 19. Schematic summary of the overall findings. The Ser/Thr kinase inhibitor PKC412 results in: (i) hypophosphorylation of non-muscle myosin heavy chain-IIA (NMHC-IIA) without impacting keratin phosphorylation, and (ii) enhances NMHC-IIA association with K8/K18 and conversion of the keratin dots into ‘normal’ wildtype (WT)-like filaments. In contrast, the Tyr kinase inhibitor PP2 leads to hypophosphorylation of K18 and K8 indirectly by first inhibiting SRC kinase which in turn leads to inhibition of other Ser/Thr kinases such as RAF. Both drugs lead to stabilization of keratin filament networks which affords protection from Fas-mediated apoptosis. While the effect of PKC412 is not sex-specific, PP2 and the RAF kinase inhibitor vemurafenib manifest a male-selective effect. pK8/K18, phospho-keratins 8 and 18.

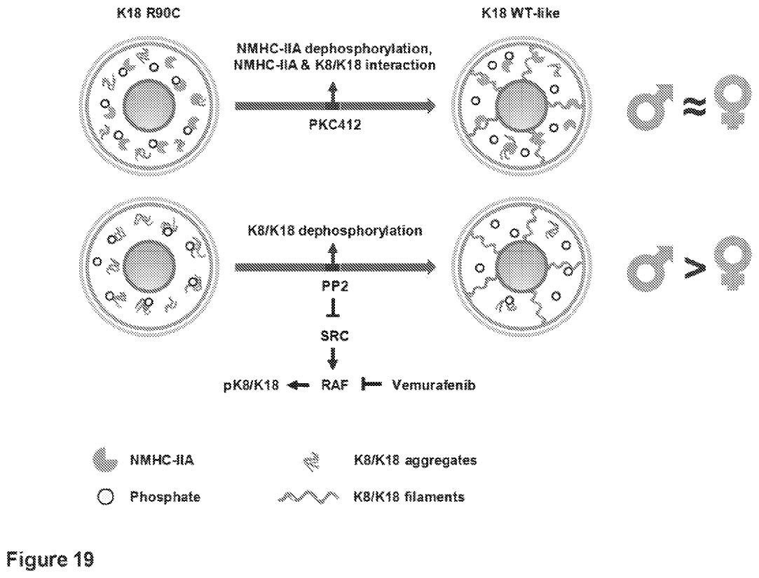

FIG. 20. PP2 reduces A549 cell density but has no effect on K18 R90C mouse liver or body weight. (A) GFP-K18 R90C transduced A549 cells were treated with DMSO or PP2 (5 μM) for 48 h. Each circle represents the relative counts of a random field. Relative cell numbers were quantified by counting random fields in the culture dish. N=3 with >200 cells/condition/experiment. ***p<0.001. (B, C) Transgenic K18 R90C mice were treated daily with DMSO or PP2 (1 mg/kg body weight; intraperitoneally) for 4d. Each circle or triangle symbol represents one mouse. PP2 treatment had no significant effect on mouse body weight or on the liver-to-body weight ratio in both female and male mice.

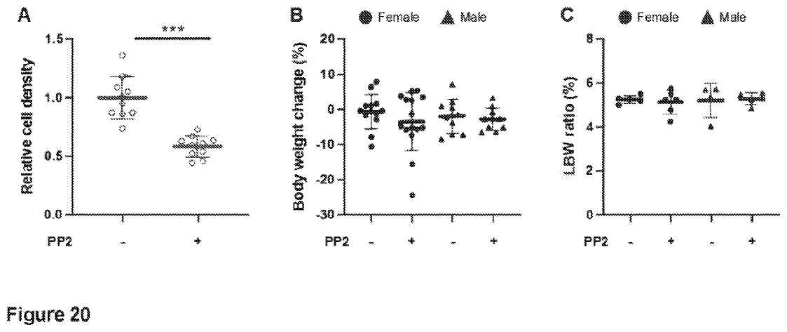

FIG. 21. PP2 protects from Fas-induced apoptosis in male but not female K18 R90C mice. Transgenic K18 R90C mice were treated daily with DMSO or PP2 (1 mg/kg body weight; intraperitoneally) for 4d then challenged with FasL (0.15 μg/g body weight; 5 h). TUNEL staining in the liver sections is visualized using an ApopTag Peroxidase In Situ Apoptosis Detection Kit (Sigma Aldrich). For quantification, see FIG. 13E (scale bar=100 m). The TUNEL-positive cells are highlighted by arrows.

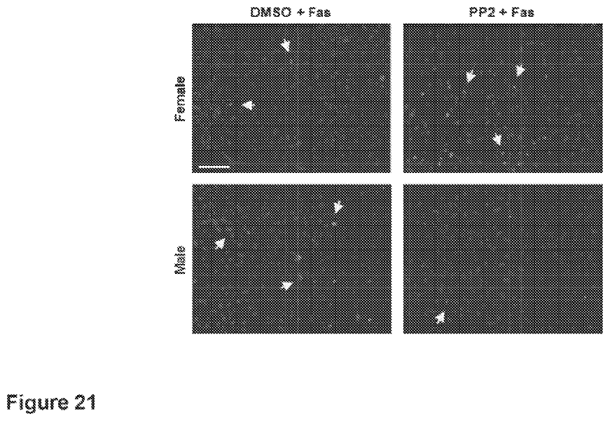

FIG. 22. PKC412 protects from Fas-induced liver injury in both male and female K18 R90C mice. K18 R90C mice were treated daily with DMSO or PKC412 (25 mg/kg body weight; intraperitoneally) for 4d then challenged with Fas antibody (0.15 μg/g body weight; 5 h). (A, B) Representative images (A) and quantification (B) of hemorrhage levels observed in hematoxylin and eosin-stained liver sections (scale bar=20 μm). Upon Fas challenge, PKC412 significantly reduced the extent of hemorrhage (B), and (C) ALT levels. Each circle (female) or triangle (male) symbol represents one mouse. *p<0.05, ***p<0.001.

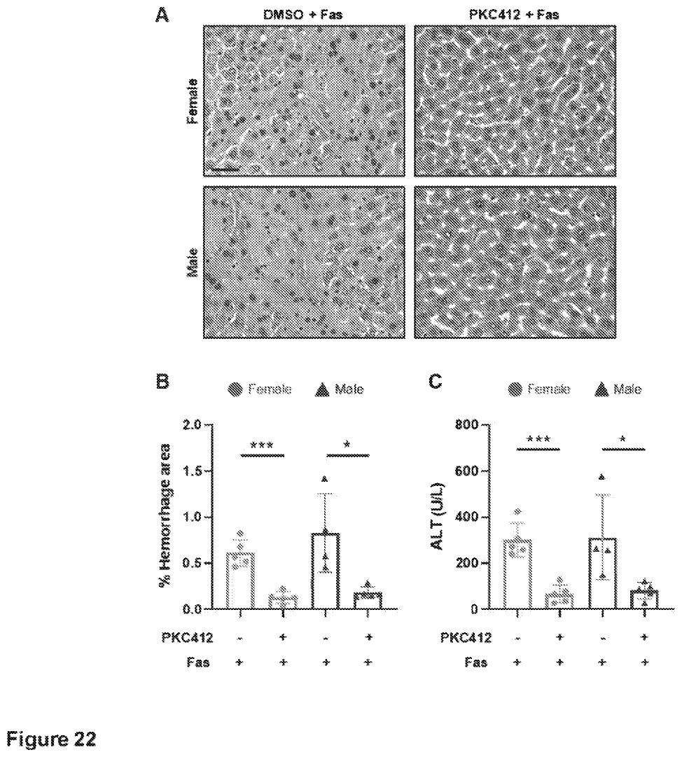

FIG. 23. PP2 inhibits SRC kinase activity and reverses hyperphosphorylation of K18 S34 and K8 S432 in cultured cells challenged with the phosphatase inhibitor okadaic acid. (A) A549 cells were treated with DMSO (vehicle) or PP2 (5 μM) for the indicated times (1, 2, 4 or 6 hours). Untreated cells were used as a negative control. (B) A549 cells were transduced with GFP-K18 R90C then treated with DMSO or PP2 (5 μM; 48 h) followed by the addition of vehicle (ethanol), or okadaic acid (OA, 1 μg/mL) to induce keratin hyperphosphorylation. Cells were lysed using 2% sodium dodecyl sulfate (SDS)-containing buffer, and the lysates were then blotted with antibodies to the indicated antigens. Coomassie staining is included as a loading control.

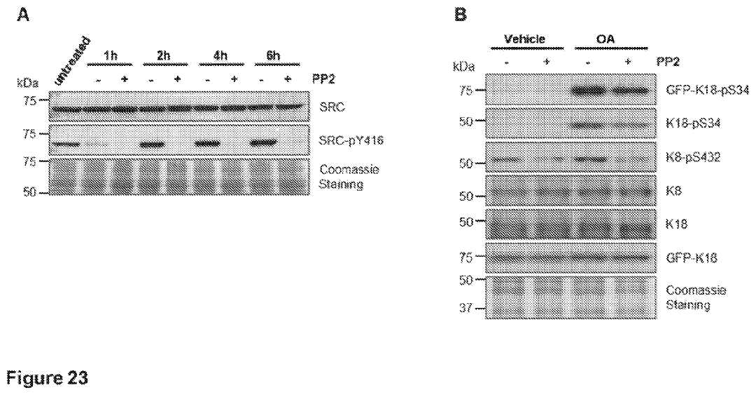

FIG. 24. Vemurafenib has no effect on K18 R90C mouse liver or body weight. Transgenic K18 R90C mice were treated daily with DMSO (−) or vemurafenib (Vem) at 1 mg/kg (+) or 5 mg/kg (++) body weight intraperitoneally for 4d then challenged with Fas antibody (0.15 μg/g body weight; 5 h). Each circle or triangle symbol represents one mouse. (A) Mouse body weight was compared between the first and the last day of treatment. (B) Mouse liver was isolated 5 h after Fas treatment and the liver-to-body weight ratio was calculated.

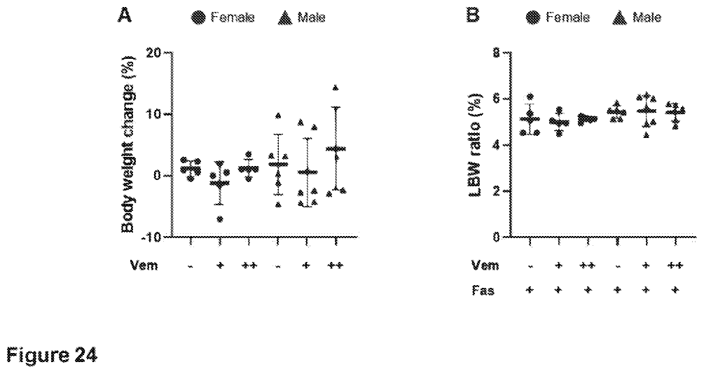

FIG. 25. The effect of PP2 on acetaminophen (APAP)-induced hepatotoxicity. Overnight-fasted K18 R90C mice were injected with APAP (50 mg/kg body weight; intraperitoneally). After 3 h, they were treated with DMSO or PP2 (1 mg/kg body weight; intraperitoneally) for 5 h. (A) Representative images of H&E-stained liver sections. Scale bar=40 μm. (B, C) Histopathological score and serum ALT level were measured to compare the effect of PP2 (+) versus vehicle (−) on APAP-induced liver injury. Each circle or triangle symbol represents one mouse. *p<0.05. (D, E) Mouse serum and liver lysates (each lane in panel E represents one mouse) were used to measure the expression of the necrosis maker (HMGB1), necroptosis maker (RIP3) and apoptosis marker (cleaved caspase 7) by Western blotting. Coomassie staining is included to show equal loading. N=2 with 3 mice/group/experiment. Relative intensities of the indicated protein bands displayed in panel D were quantified (E). Each circle or triangle symbol represents one mouse. *p<0.05, **p<0.01. (F) Schematic to highlight the types of APAP-induced hepatocyte injury.

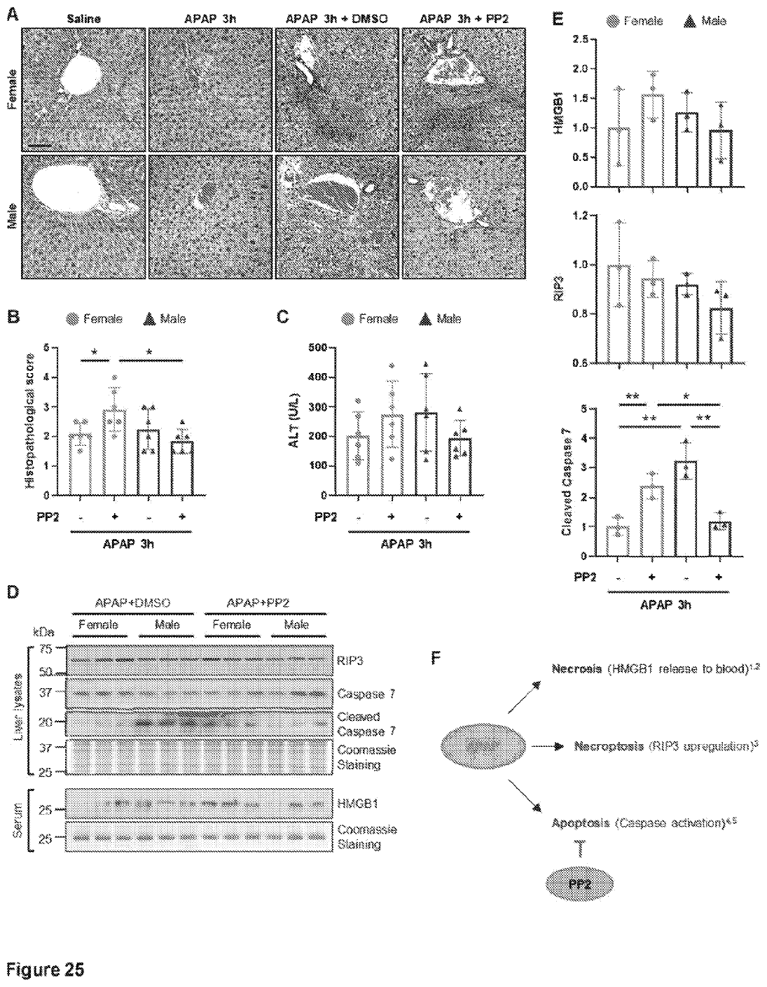

FIG. 26. UPLC-UV method for detecting PP2 at picomole levels. (A) Structure of PP2. (B) Representative chromatograms of PP2 (400 picomole) and blank injection, recorded at 254 nm. (C) UV-visible spectra of the eluting peak shown in panel B. (D) Retention times of different picomole amounts of PP2 injections. For each picomole amount tested, three repeat injections were performed, and the entire set was repeated on a different day (displayed in the column on the far right of the table). The limit of detection (LOD) and limit of quantitation (LOQ) for the assay are shown. (E) Plot of area under the curve (AUC) of PP2 peak versus PP2 mass (in picomoles) shows a linear correlation. (F) Solid phase extraction of fetal bovine serum (FBS) spiked with PP2 shows no matrix effect. ns=p>0.05.

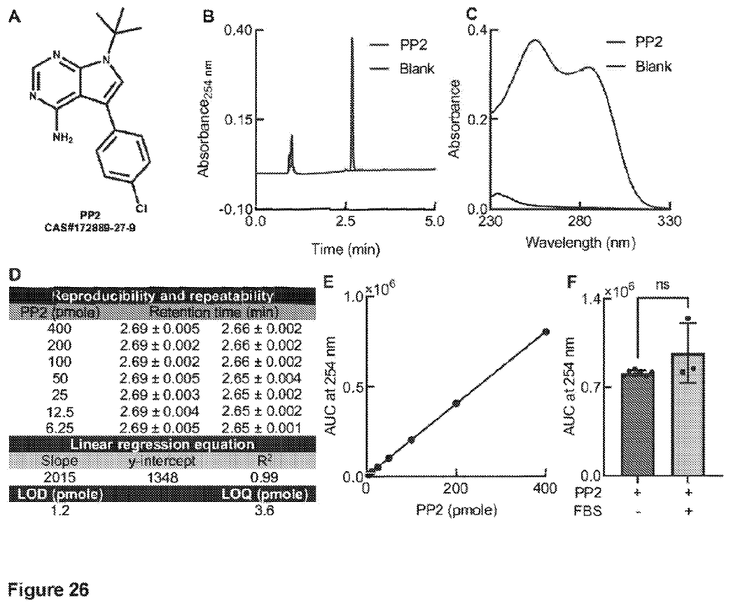

FIG. 27. (A) GFP-K18 R90C lentivirus-transduced A549 cells were treated with DMSO (−), PKC412 (0.8 μM), PP2 (5 μM), PKC412 (0.4 μM)+PP2 (2.5 μM), PKC412 (0.8 μM)+PP2 (5 μM) for 48 h then treated with IFN-7 (40 ng/mL, 6 h) then Fas ligand (FasL) (100 ng/mL, 12 h) to induce apoptosis. Cell lysates were analyzed by blotting with antibodies to the indicated apoptosis markers. Actin blot is included as a loading control. (B) Relative intensity of the indicated bands from 3 individual experiments was quantified. Each circle represents a separate experiment. #p<0.05, ##p<0.01, ###p<0.001 when comparing with DMSO treatment. *p<0.05 when comparing PKC412 (0.8 μM)+PP2 (5 μM) with PKC412 or PP2 alone. Comparisons were done using the unpaired Student's t-test. Data show mean±standard deviation (SD).

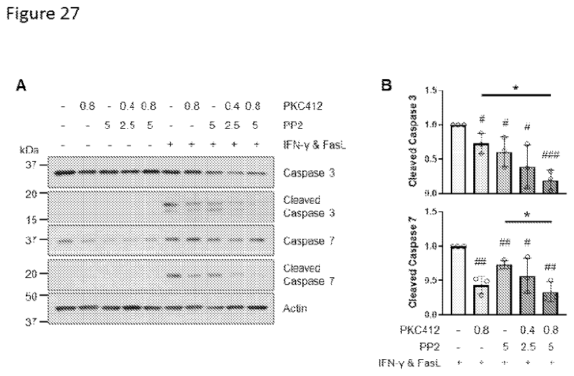

FIG. 28. Transgenic K18 R90C mice (8-10 wks old, male) were treated daily with DMSO (−), PKC412 (25 mg/kg), PP2 (1 mg/kg), vemu-rafenib (Vem, 1 mg/kg), PKC412 (12.5 mg/kg)+PP2 (0.5 mg/kg) or PKC412 (12.5 mg/kg)+Vem (0.5 mg/kg ip for 4d then challenged with Fas antibody (0.25 μg/g body weight; 5 h). (A, B) Representative images and quantification of the hemorrhage observed in hematoxylin and eosin-stained liver. Scale bar=20 μm. The hemorrhage score was calculated using QuPath and ImageJ software. (C) Serum ALT levels were measured using the Comprehensive Diagnostic Profile Rotors (Abaxis). For panels B and C, each circle symbol represents one mouse. The # indicates significant difference compared with the untreated and DMSO groups, respectively. *shows significant difference between various treatment groups. Comparisons were done using the Mann-Whitney U test. Data are expressed as mean±standard deviation (SD). #,*p<0.05, ##,**p<0.01, ###,***p<0.001, ####,****p<0.0001.

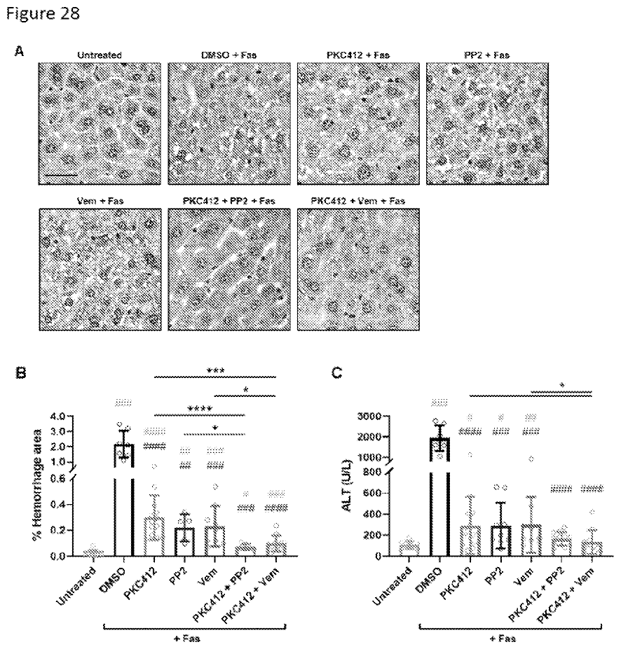

FIG. 29. (A) PKC412 reduced keratin aggregates in K14 R125C cells as depicted by K5 staining. Cells were cultured with DMSO or 0.8 μM PKC412 for 24 h and analyzed by immunofluorescence microscopy. (B) Quantification of the keratin aggregate-containing cells treated with PKC412 (mean=13%) compared to DMSO-treated cells (mean=56%; median is shown, 2-tailed Student's t-test, n=3 with >100 counted cells/experiment,***p<0.001). (C) K14 R125C cells were cultured in DMSO (1 μM), 2.5 μM Vem (Vem) alone or 2.5 μM Vem+0.4 μM PKC412 for 24 h then analyzed by fluorescence microscopy (K5 staining). Scale bar=50 μm, detail=10 μm (for panels A,C). (D) Panel shows the changes of K14 R125C keratinocytes treated with Vem (mean for 1 μM=56% and 2.5 μM=46%) or in combination with PKC412 (mean=46%) compared with DMSO-treated EBS cells (mean for DMSO=78%; median is shown, 2-way ANOVA, Tukey's multiple comparisons test, n=1, 10 images with >150 counted cells, *p>0.05, ***p>0.001).

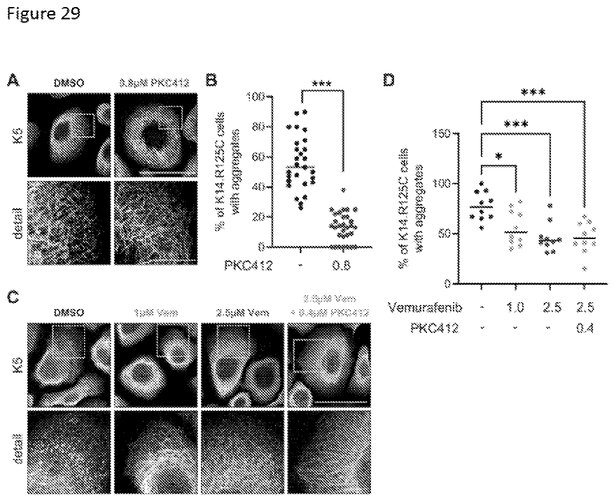

### DETAILED DESCRIPTION

Keratins are intermediate filament proteins (IFs) whose dysfunction is associated with an extensive group of human diseases. Keratins exist as obligate noncovalent type-I/type-II heteropolymers, including keratins 8 and 18 (K8/K18) in hepatocytes (and other glandular single layered epithelial cells) and keratins 5 and 14 (K5/K14) in basal keratinocytes. Epidermolysis bullosa simplex (EBS) was the first human disease to be associated with IF mutations; and is caused in its most severe form by mutation at the highly conserved arginine (K14 R125C) that markedly perturbs the K5/K14 filament networks in keratinocytes. A homologous arginine mutation to K14 R125C, K18 R90C, when introduced as a transgene in mice, results in hepatocyte keratin filament disruption and aggregation, keratin hyperphosphorylation, and predisposition to Fas-induced among several other types of liver injury. Of clinical significance, mutations at conserved residues, including K18 D89H and K8 K393R that also lead to disruption of keratin filaments when tested in culture systems, have been reported among the most severe cases of drug-induced liver injury. Inducing keratin hyperphosphorylation by phosphatase inhibitors also leads to similar disruption of keratin filament into dots. In humans, K8 and K18 variants predispose their carriers to liver disease progression, with unique variant association with specific races and ethnicities. This contrasts with the highly penetrant epidermal keratin mutations that cause rather than predispose to human disease. As such, human K8/K18 variants serve as the ‘first hit’, with the ‘second hit’ being an underlying acute or chronic liver disease (e.g., metabolic, viral or toxin-related ‘second hits’). Therefore, the presence to K8/K18 variants associates with poor outcomes including the need for liver transplantation or death from the liver disease.

One critical unmet need for the more than 70 IF-associated diseases is the lack of directed therapies. The majority of therapeutic approaches for treating keratinopathies have focused on allele-specific gene silencing or ablation and on stabilization of the IF network by small-molecule compounds. Given that disruption of keratin organization predisposes hepatocytes to apoptosis and necrosis, a high-throughput drug screening of kinase inhibitors that normalize keratin filaments was conducted. Upon deployment of this cell-based drug-screening approach, the Ser/Thr kinase inhibitor PKC412 was identified and shown to revert disrupted keratin aggregates to wildtype-like extended filament networks and to protect mice carrying the K18 R90C mutation from Fas-mediated liver injury. Although K8/K18 become hyperphosphorylated on serine residues upon K18 R90C mutation, PKC412 leads to a protective effect by inducing hypophosphorylation of the non-muscle myosin heavy chain-IIA (NMHC-IIA) protein (without changes at major keratin phospho-sites), which facilitates the binding of NMHC-IIA to keratins and consequent stabilization the filament network. The use of kinase inhibitors has also been successful in protecting animals from cardiomyopathy caused by mutations in the nuclear IF lamin A/C. Accordingly, effective therapeutic treatments are needed.

It was hypothesized that EBS-associated keratin mutations promote posttranslational modifications (PTMs), particularly phosphorylation, in keratins or keratin-associated proteins to enhance disease severity. As described herein, this hypothesis was tested by treating EBS-associated keratinocytes with the multi-kinase inhibitor PKC412. Functional assays in combination with phosphoproteomic analysis revealed that PKC412 promoted the reformation of an intact keratin cytoskeleton from aggregates by altering keratin and desmoplakin phosphorylation in EBS keratinocytes. As such, PKC412 represents a treatment for counteracting the clinical manifestations of EBS.

As used herein, the following terms have the meaning ascribed to them unless specified otherwise.

The terms “treat”, “treatment”, or “treating” to the extent it relates to a disease or condition includes inhibiting the disease or condition, eliminating the disease or condition, and/or relieving one or more symptoms of the disease or condition. The terms “treat”, “treatment”, or “treating” also refer to both therapeutic treatment and/or prophylactic treatment or preventative measures, wherein the object is to prevent or slow down (lessen) an undesired physiological change or disorder. For example, beneficial or desired clinical results include, but are not limited to, alleviation of symptoms, diminishment of extent of disease or disorder, stabilized (e.g., not worsening) state of disease or disorder, delay or slowing of disease progression, amelioration or palliation of the disease state or disorder, and remission (whether partial or total), whether detectable or undetectable. Those in need of treatment include those already with the disease or disorder as well as those prone to have the disease or disorder or those in which the disease or disorder manifestations are to be prevented. In one embodiment “treat”, “treatment”, or “treating” does not include preventing or prevention.

The phrase “therapeutically effective amount” or “effective amount” includes but is not limited to an amount of a compound of the that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

“Systemic delivery,” as used herein, refers to delivery that leads to a broad biodistribution within an organism. Some techniques of administration can lead to the systemic delivery of certain agents, but not others. Systemic delivery means that a useful, preferably therapeutic and nontoxic, amount of an agent is exposed to most parts of the body. To obtain broad biodistribution generally requires a blood lifetime such that the agent is not rapidly degraded or cleared (such as by first pass organs (liver, lung, etc.) or by rapid, nonspecific cell binding or uptake) before reaching a disease site distal to the site of administration. Systemic delivery can be by any means known in the art including, for example, intravenous, subcutaneous, and intraperitoneal.

“Local delivery,” as used herein, refers to delivery directly to a target site within an organism, e.g., to a localized area of skin using a cream or ointment formulation using a formulation for topical administration.

As used herein, the term “aqueous solution” refers to a composition comprising in whole, or in part, water.

The pharmaceutical compositions of the present invention may be sterilized by conventional, well-known sterilization techniques. Aqueous solutions can be packaged for use or filtered under aseptic conditions. The compositions can contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, and calcium chloride.

For in vivo administration, administration can be in any manner known in the art, e.g., by injection, oral administration, inhalation (e.g., intransal or intratracheal), transdermal application, or rectal administration. Administration can be accomplished via single or divided doses. The pharmaceutical compositions can be administered parenterally, i.e., intraarticularly, intravenously, intraperitoneally, subcutaneously, or intramuscularly. In some embodiments, the pharmaceutical compositions are administered intravenously or intraperitoneally by a bolus injection (see, e.g., U.S. Pat. No. 5,286,634). Intracellular nucleic acid delivery has also been discussed in Straubringer et al., Methods Enzymol., 101:512 (1983); Mannino et al., Biotechniques, 6:682 (1988); Nicolau et al., Crit. Rev. Ther. Drug Carrier Syst., 6:239 (1989); and Behr, Acc. Chem. Res., 26:274 (1993). The compounds and compositions can be administered by direct injection at the site of disease or by injection at a site distal from the site of disease (see, e.g., Culver, HUMAN GENE THERAPY, MaryAnn Liebert, Inc., Publishers, New York. pp. 70-71 (1994)). The disclosures of the above-described references are herein incorporated by reference in their entirety for all purposes.

In certain embodiments, the pharmaceutical compositions may be delivered by intranasal sprays, inhalation, and/or other aerosol delivery vehicles. Methods for delivering nucleic acid compositions directly to the lungs via nasal aerosol sprays have been described, e.g., in U.S. Pat. Nos. 5,756,353 and 5,804,212. Likewise, the delivery of drugs using intranasal microparticle resins and lysophosphatidyl-glycerol compounds (U.S. Pat. No. 5,725,871) are also well-known in the pharmaceutical arts. Similarly, transmucosal drug delivery in the form of a polytetrafluoroetheylene support matrix is described in U.S. Pat. No. 5,780,045. The disclosures of the above-described patents are herein incorporated by reference in their entirety for all purposes.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions are preferably administered, for example, by intravenous infusion, orally, topically, intraperitoneally, intravesically, or intrathecally.

Generally, when administered intravenously, the formulations are formulated with a suitable pharmaceutical carrier. Many pharmaceutically acceptable carriers may be employed in the compositions and methods of the present invention. Suitable formulations for use in the present invention are found, for example, in REMINGTON'S PHARMACEUTICAL SCIENCES, Mack Publishing Company, Philadelphia, Pa., 17th ed. (1985). A variety of aqueous carriers may be used, for example, water, buffered water, 0.4% saline, 0.3% glycine, and the like, and may include glycoproteins for enhanced stability, such as albumin, lipoprotein, globulin, etc. Generally, normal buffered saline (135-150 mM NaCl) will be employed as the pharmaceutically acceptable carrier, but other suitable carriers will suffice. These compositions can be sterilized by conventional liposomal sterilization techniques, such as filtration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc. These compositions can be sterilized using the techniques referred to above or, alternatively, they can be produced under sterile conditions. The resulting aqueous solutions may be packaged for use or filtered under aseptic conditions and lyophilized, the lyophilized preparation being combined with a sterile aqueous solution prior to administration.

In certain applications, administration of the active compound(s) is via oral administration to an individual. The compounds may be incorporated with excipients and used in the form of ingestible tablets, buccal tablets, troches, capsules, pills, lozenges, elixirs, mouthwash, suspensions, oral sprays, syrups, wafers, and the like (see, e.g., U.S. Pat. Nos. 5,641,515, 5,580,579, and 5,792,451, the disclosures of which are herein incorporated by reference in their entirety for all purposes). These oral dosage forms may also contain the following: binders, gelatin; excipients, lubricants, and/or flavoring agents. When the unit dosage form is a capsule, it may contain, in addition to the materials described above, a liquid carrier. Various other materials may be present as coatings or to otherwise modify the physical form of the dosage unit. Of course, any material used in preparing any unit dosage form should be pharmaceutically pure and substantially non-toxic in the amounts employed.

The methods of the present invention may be practiced in a variety of hosts. Preferred hosts include mammalian species, such as primates (e.g., humans and chimpanzees as well as other nonhuman primates), canines, felines, equines, bovines, ovines, caprines, rodents (e.g., rats and mice), lagomorphs, and swine.

PKC412 (midostaurin) is a multi-targeted protein kinase inhibitor that has been investigated for the treatment of acute myeloid leukemia (AML), myelodysplastic syndrome (MDS) and advanced systemic mastocytosis. It is a semi-synthetic derivative of the protein kinase C inhibitor, staurosporine, an alkaloid from the bacterium Streptomyces staurosporeus (4′-N-benzoylstaurosporine), that has been recommended for oral administration (e.g., 50 mg orally twice daily with food).

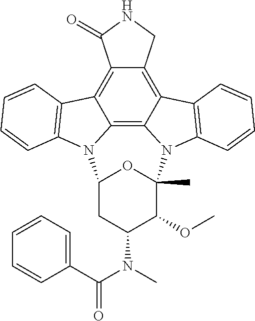 [SMILES: CO[C@@H]1[C@H](N(C)C(=O)c2ccccc2)C[C@H]2O[C@]1(C)n1c3ccccc3c3c4c(c5c6ccccc6n2c5c31)C(=O)NC4]

In certain embodiments, other derivatives of staurosporine may be used in a similar manner to treat EBS and related conditions (see, e.g., WO 03/037347 and WO 2004/112794, the disclosure of which are specifically incorporated by reference).

In certain embodiments, PKC412 can be used to treat other diseases and conditions including other keratin skin diseases, e.g., caused by mutations in other epidermal keratins including keratin pairs 1/10, 6/16/17, e.g., to reduce keratin filament aggregation, e.g., in a patient in need thereof.

In certain embodiments, any of the following can be treated:
Epidermolytic hyperkeratosis (OMIM #113800);
Epidermolytic palmoplantar keratoderma (OMIM #144200);
Palmoplantar keratoderma, nonepidermolytic (OMIM #600962);
Pachyonychia congenita type 1 (OMIM #167200); or
Pachyonychia congenita type 2 (OMIM #167210).

Hepatocyte keratin polypeptides 8/18 (K8/K18) are unique among intermediate filaments proteins (IFs) in that their mutation predisposes to, rather than causes, human disease. Mice that overexpress human K18 R90C manifest disrupted hepatocyte keratin filaments with hyperphosphorylated keratins and predisposition to Fas-induced liver injury. It was hypothesized that high-throughput screening will identify compounds that protect the liver from mutation-triggered predisposition to injury.

Using A549 cells transduced with a lentivirus K18 construct and high-throughput screening, the SRC-family tyrosine kinases inhibitor, PP2, was identified as a compound that reverses keratin filament disruption and protects from apoptotic cell death caused by K18 R90C mutation at this highly conserved arginine. PP2 ameliorated Fas-induced apoptosis and liver injury in male but not female K18 R90C mice. The PP2 male selectivity is due to its lower turnover in male versus female livers. Knockdown of SRC but not another kinase target of PP2, protein tyrosine kinase-6, in A549 cells abrogated the hepatoprotective effect of PP2. Phosphoproteomic analysis and validation showed that the protective effect of PP2 associates with Ser/Thr but not Tyr keratin hypophosphorylation, and differs from the sex-independent effect of the Ser/Thr kinase inhibitor PKC412. Inhibition of RAF kinase, a downstream target of SRC, by vemurafenib had a similar protective effect to PP2 in A549 cells and male K18 R90C mice.

The structures of PP2 and vemurafenib are provided below.

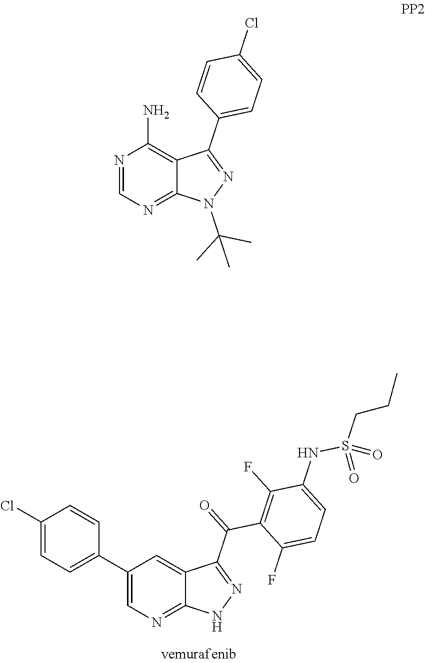 [SMILES: CC(C)(C)n1nc(-c2ccc(Cl)cc2)c2c(N)ncnc21 ; CCCS(=O)(=O)Nc1ccc(F)c(C(=O)c2n[nH]c3ncc(-c4ccc(Cl)cc4)cc23)c1F]

PP2 protects, in a mouse male-selective manner, keratin mutation-induced liver injury by inhibiting SRC-triggered downstream Ser/Thr phosphorylation of K8/K18, which is phenocopied by the RAF kinase inhibitor, vemurafenib. The PP2/vemurafenib-associated findings, and their unique mechanisms of action, further support the potential role of select kinase inhibition as therapeutic opportunities for keratin and other IF-associated human diseases.

The term “animal” includes mammalian species, such as a human, mouse, rat, dog, cat, hamster, guinea pig, rabbit, livestock, and the like.

The term “salts” includes any anionic and cationic complex. Non-limiting examples of anions include inorganic and organic anions, e.g., hydride, fluoride, chloride, bromide, iodide, oxalate (e.g., hemioxalate), phosphate, phosphonate, hydrogen phosphate, dihydrogen phosphate, oxide, carbonate, bicarbonate, nitrate, nitrite, nitride, bisulfite, sulfide, sulfite, bisulfate, sulfate, thiosulfate, hydrogen sulfate, borate, formate, acetate, benzoate, citrate, tartrate, lactate, acrylate, polyacrylate, fumarate, maleate, itaconate, glycolate, gluconate, malate, mandelate, tiglate, ascorbate, salicylate, polymethacrylate, perchlorate, chlorate, chlorite, hypochlorite, bromate, hypobromite, iodate, an alkylsulfonate, an arylsulfonate, arsenate, arsenite, chromate, dichromate, cyanide, cyanate, thiocyanate, hydroxide, peroxide, permanganate, and mixtures thereof.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

When a bond in a compound formula herein is drawn in a non-stereochemical manner (e.g., flat), the atom to which the bond is attached includes all stereochemical possibilities. Unless otherwise specifically noted, when a bond in a compound formula herein is drawn in a defined stereochemical manner (e.g., bold, bold-wedge, dashed or dashed-wedge), it is to be understood that the atom to which the stereochemical bond is attached is enriched in the absolute stereoisomer depicted. In one embodiment, the compound may be at least 51% the absolute stereoisomer depicted. In another embodiment, the compound may be at least 60% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95% the absolute stereoisomer depicted. In another embodiment, the compound may be at least 99% the absolute stereoisomer depicted. Unless stated otherwise herein, the term “about”, when used in connection with a value or range of values, means plus or minus 5% of the stated value or range of values.

The present invention will be described in greater detail by way of specific examples. The following examples are offered for illustrative purposes and are not intended to limit the invention in any manner. Those of skill in the art will readily recognize a variety of noncritical parameters which can be changed or modified to yield essentially the same results.

Certain aspects of the present invention are also exemplified and described in Rietscher et al., Journal of Investigative Dermatology, 142, 3282-3293 (2022), including Supplementary material https://doi.org/10.1016/j.jid.2022.05.1088 and Li et al., Hepatology, 77, 144-158 (2023), https://doi.org/10.1002/hep.32574, both of which publications are explicitly incorporated by reference herein.

### EXAMPLES

### Example 1. Kinase Inhibition Prevents Epithelial Sheet Damage in Autosomal Dominant Epidermolysis Bullosa Simplex Via Keratin and Cell Contact Stabilization

Immunofluorescence microscopy was used to examine the impact of six mutations in either K5 or K14 on keratin filament (KF) organization in immortalized patient derived EBS keratinocytes. Normal human keratinocytes (NHK) displayed an intact keratin cytoskeleton at 37° C., whereas K14.R125C and K14.R125G keratinocytes revealed keratin aggregates localized preferentially at the cell periphery. In contrast, K14.Y415H, K14.N123S, K5.G138E/L175F (double mutant) and K5.E477D mutants, which also cause EBS-S, led to KF reorganization but not aggregation under standard culture conditions. Notably, densely packed keratin bundles were oriented perinuclearly in cells expressing K14.N123S, while K5.E477D-expressing keratinocytes displayed predominantly short KF at the cell periphery that mostly aligned in parallel to the cell center. Thus, different patient-derived K5 and K14 mutations displayed a distinct, mutation-specific impact on KF organization at 37° C.

The impact of elevated temperature as a disease-related stress parameter on KF organization in cells was also investigated. In NHK, increasing the temperature to 42° C. for 15 min led to modest KF reorganization into bundles, which recovered into typical KF after 30 min. In contrast, almost all EBS-associated cell lines formed aggregates immediately following exposure to elevated temperature. K14.N123S keratinocytes were the one exception that instead of aggregation displayed extensive perinuclear keratin bundles at 42° C. compared to 37° C. The keratin aggregates were resolved into the initial KF organization after 30 min at 37°. These data indicated that upon mild heat stress, most EBS-S-associated keratin mutations were unable to sustain a stress-resilient cytoskeleton. Because mutation of the site K14.R125 to cysteine resulted in prominent keratin aggregates already under normal culture conditions, the K14.R125C cell line was used for most subsequent experiments.

It was hypothesized that EBS-associated mutations promote keratin and/or keratin-associated protein phosphorylation to act in concert and enhance disease severity. It was further postulated that interfering with major kinases implicated in phosphorylation of keratins and/or keratin-associated proteins would reduce keratin aggregation/reorganization. To test these hypotheses, the multi-kinase inhibitor PKC412 was used, known to inhibit particularly PKA, PKC, or AKT among others, and its effect were examined on EBS-associated keratin aggregates by immunofluorescence staining. Notably, compared to vehicle-treated cells, PKC412 strongly reduced keratin aggregates in K14.R125C keratinocytes by about 40%.

The impact of PKC412 on intercellular cohesion of K14.R125C keratinocytes was also examined in comparison to NHK. PKC412-treated EBS monolayers remained fully intact in comparison to vehicle-treated K14.R125C monolayers, indicating that PKC412 efficiently improved the cell-sheet stability of EBS keratinocytes by restoring an intact keratin cytoskeleton and/or strengthening keratin-desmosome interactions. Consistently, PKC412 treatment also rescued fragility of K14.R125G mutant sheets, indicating that PKC412 can be used more broadly to treat keratin defects associated with dominant mutations.

To substantiate the efficacy of PKC412 on EBS-associated keratinocytes, real-time impedance spectroscopy measurements were employed using multi-electrode arrays (MEAs). This assay investigates the functionality of intercellular junctions and acquisition of an epithelial barrier. NHK and EBS keratinocytes established a similar relative impedance, when cultured in low calcium medium (LCM). However, after increasing the calcium concentration in the medium (high calcium medium, HCM) to induce intercellular adhesion, K14.R125C keratinocytes failed to establish the same relative impedance to levels observed for NHK. Thus, impedance spectroscopy enabled quantification of cell-cell adhesion differences and to define differences between EBS and control cells.

Upon PKC412 treatment, EBS keratinocytes showed a significant and concentration-dependent increase in impedance readings. At 1 μM, the relative impedance of EBS keratinocytes reached values equal to control cells, whereas concentrations above 3 μM of PKC412 had no apparent additional benefit. Control cells also showed an increased impedance, pointing to a broad effect of PKC412. Moreover, concentration response curves revealed EC₅₀ values of 157 nM for K14.R125C cells and 84 nM for NHK (24 h treatment), suggesting a more sensitive response of control cells. It was concluded that PKC412 significantly improved the KF state in K14.R125C cells and restored intercellular adhesion to an extent that was highly similar to that of a normal non-mutant state.

The reorganization of the KF network is influenced by various PTMs including phosphorylation, acetylation or sumoylation, modifications that participate in a complex crosstalk to fine-tune keratin properties and their impact on cellular functions. Because the EBS K14.R125C mutation was associated with altered keratin phosphorylation, it was hypothesized that changes in phosphorylation are one mechanism to target aggregated KF that contribute to the pathogenesis of EBS. To get an unbiased view on the impact of this mutation on the phosphorylation of keratins and non-keratin proteins, an in silico prediction was performed restricted to K14 phosphorylation, based on a recently described prediction tool (Lin et al., (2020) Genotype-phenotype analysis of LMNA-related diseases predicts phenotype-selective alterations in lamin phosphorylation. FASEB J 34:9051-73). For the two mutations K14.R125C and K14.R125G, reduced phosphorylation at K14.S128 was predicted. Next, the Mutation IMpact on Phosphorylation (MIMP) prediction was performed to predict candidate kinases that might act on this site. MIMP predicted that substitutions at R125 should prevent phosphorylation by numerous kinases, particularly AGC-kinases (e.g., PKA, PKCf). Whether the predicted K14 site is in fact phosphorylated may be affected by the presence of K5, the obligatory heterodimer partner of K14, an issue not addressed by MIMP.

To address the predicted phosphorylation changes experimentally, immunoblotting of total lysates from NHK and K14.R125C keratinocytes was performed using a phospho-specific antibody that recognizes the AGC-kinase motif RXXpS/pT. In contrast to the bioinformatic prediction, EBS cell lysates revealed significantly increased phosphorylation compared to NHK, indicating that keratins and/or keratin-associated proteins showed increased phosphorylation at AGC-kinase motifs in the presence of EB mutations. Next, the effect of PKC412 treatment on AGC-kinase phosphorylation sites was tested using this antibody. Indeed, PKC412-treated EBS cells showed a significant reduction in phosphorylation compared to vehicle-treated cells. These findings indicate that PKC412 inhibited AGC-kinases responsible for keratin or keratin-associated protein phosphorylation, thereby stabilizing KF organization.

To address the molecular mechanism by which PKC412 improves the stress resilience of EBS keratinocytes, a global quantitative phosphoproteomic approach was employed to analyze broad changes in protein phosphorylation upon PKC412. Gene ontology (GO) term analysis revealed enrichment in biological processes related to cell junction organization and assembly, hemidesmosome assembly and cytoskeleton organization. Furthermore, motif-enrichment analysis revealed that PKC412 treatment significantly reduced phosphorylation at ERK1/2, GSK-3 and CDK5 kinase motifs. It was examined whether PKC412 treatment directly affected keratin phosphorylation and observed altered phosphorylation of the basal keratins K5 and K14 as well as of wound-healing keratins K6A, K6B, K16 and K17. For further analysis, sites showing reduced phosphorylation upon PKC412 treatment were investigated and observed lower phosphorylation of K5 at two threonine and 17 serine residues, preferentially located in the head and tail domain of K5. From these, K5-T24 phosphorylation was reported for mitotic cells. A reduction in phosphorylation was observed at one threonine and 13 serine residues within K14. K6A also manifested reduced phosphorylated at one tyrosine and nine serine residues, whereas no significant reduction in phosphorylation in K6B and K16 was detected upon PKC412 treatment in comparison to vehicle-treated cells. K17 phosphorylation was reduced at eight serine residues, exclusively located in the head and tail domain of K17. From all detected phosphorylation sites, four serine residues (K5 P-S6, K6A P-S12 and P-S19, and K17 P-S44) are located in the RXXpS motif, in good agreement with the reduced phosphorylation of RXXpS motifs upon PKC412 treatment shown by immunoblotting. To validate key phospho-proteome results by immunoblotting of total cell lysates from PKC412- or vehicle-treated NHK and K14.R125C cells, a phospho-site specific antibody recognizing K17 at P-S44 was used. PKC412 treatment significantly reduced K17 phosphorylation at this site. These findings indicate that PKC412 inhibited kinases directly responsible for keratin phosphorylation and thereby promoted reformation of KF organization.

The increased intercellular adhesion of EBS keratinocytes upon PKC412 treatment led to the hypothesis that beyond keratins, phosphorylation events in keratin-associated proteins promoted the stability of K14.R125C epithelial sheets. The investigation was therefore focused on desmoplakin (DSP), which links KFs to the desmosomal plaque to enhance intercellular cohesion and mechanical resilience. A phospho-proteomic analysis discovered altered phospho-sites in DSP. As before, the investigation focused on reduced phospho-sites and found less phosphorylation at six serine residues within the amino-terminal domain of DSP and at nine serine residues within the carboxy-terminal keratin-binding domain of DSP. Two of those identified reduced phospho-sites in DSP were validated by immunoblotting total cell lysates of NHK and K14.R125C keratinocytes using a phospho-specific DSP antibody, which recognizes P-DSP at S165 and S166. PKC412 treatment significantly reduced phosphorylation of DSP at these sites in comparison to DMSO-treated cells. To check whether PKC412-induced altered DSP phosphorylation leads to its relocalization, immunofluorescence microscopy was performed. NHK and K14.R125C keratinocytes were cultured with PKC412 or DMSO in the presence of 1.2 mM CaCl₂ to induce cell-cell contacts and stained for K5 and DSP. A significant increase of DSP was observed at sites of cell-cell contacts upon PKC412 treatment compared to vehicle-treated cells, suggesting increased intercellular cohesion by strengthened desmosome formation. In addition to altered DSP phosphorylation in its amino-terminal domain, which is critical for binding desmosomal plaque proteins such as plakoglobin or plakophilins, several altered phospho-sites in the DSP carboxy-terminal domain were identified. Among these sites, P-DSP S2849 regulates keratin association and desmosome adhesive strength. Notably, the P-deficient mutant DSP-S2849G effectively increased intercellular adhesion because of its higher KF anchorage. Probing this site with a P-specific antibody showed that PKC412 treatment resulted in a significantly reduced DSP phosphorylation at S2849 in comparison to DMSO-treated cells. These results indicate that PKC412 inhibited kinases that phosphorylate keratins and DSP, thereby strengthening the anchorage of KFs to DSP and the desmosomal plaque. Collectively, these events explain the increased intercellular adhesive strength of EBS K14.R125C keratinocytes revealed by epithelial sheet and impedance spectroscopy assays.

Using phospho-mimetic and phospho-deficient mutants, it was found that hyperphosphorylation aggravated EBS-associated keratin aggregation, whereas hypophosphorylation stimulated formation of a more normal keratin cytoskeleton. Based on these data, it was investigated whether the multi-kinase inhibitor PKC412, an oral US Food and Drug Administration-approved drug for the treatment of acute myeloid leukemia (AML) and for advanced systemic mastocytosis (SM), is effective in a keratinocyte model of EBS. It is show herein that PKC412: (i) significantly reduced KF aggregation in K14.R125C EBS-associated keratinocytes, (ii) strongly improved the shear-stress resilience of epithelial sheets formed by these cells, (iii) increased impedance using a real-time impedance spectroscopy assay, and (iv) affected phosphorylation of keratins and of the desmosomal linker protein desmoplakin.

Employing phosphoproteomic analysis, it was found that PKC412 treatment directly affected phosphorylation of keratins K5 and K14 and the wound healing keratins K6A and K17, which potentially allow a shift from keratin aggregates to filaments. Of note, phosphorylation of K17 at S44 was decreased upon PKC412 treatment and observed by proteomic analysis and validated by immunoblotting. Interestingly, motif enrichment analysis of the phospho-proteome analysis revealed decreased ERK1/2 phospho-sites upon PKC412 treatment, suggesting the involvement of ERK signaling. Further, in addition to directly modulating keratin phosphorylation, PKC412 might affect keratin aggregation indirectly by acting on keratin-associated proteins.

Given the strongly increased intercellular adhesion of K14.R125C keratinocytes upon PKC412 treatment, as revealed by the dispase assay, focus was made on DSP, and it was observed reduced phosphorylation at S165 and S166 upon PKC412 treatment coupled with increased localization of DSP at cell-cell contacts. Although not detected in the phospho-proteome analysis, DSP S2849 phosphorylation was also significantly reduced upon PKC412 treatment.

The data presented herein strongly support the hypothesis that EBS-associated keratin mutations alter PTMs, in particular phosphorylation, and that subsequent aggravation of keratin aggregation-dependent skin pathology can be reversed by drugs such as PKC412. This will allow the reformation of a more stress-resilient keratin cytoskeleton along with improved skin integrity in response to environmental stress. It is hypothesized that PKC412-induced reduction of keratin aggregates, which compromise keratinocyte viability, represents a viable strategy for the treatment of EBS.

As PKC412 is currently in use for advanced systemic mastocytosis and AML, these findings provide a potential clinical translation and repurposing of PKC412 for use in patients with EBS-S. Originally, PKC412 was developed as a protein kinase C inhibitor for the treatment of patients with solid tumors. On the basis of preclinical studies, a phase 1b study involving patients with newly diagnosed AML was conducted. This study revealed that oral PKC412 could be administered safely with an acceptable side-effect profile at a dose of 50 mg twice daily. In a study by He et al. pharmacokinetics, mass balance, absorption, metabolism, and excretion of PKC412 were determined in healthy volunteers showing that the drug was well tolerated after a single oral dose (50 mg) formulated as a microemulsion. In sum, PKC412 has shown good tolerability among patients in the completed clinical trials. An oral PKC412 administration may be appropriate for generalized severe EBS. It is additionally proposed herein that a formulation for topical, local PKC412 administration such as a cream or ointment for patients with localized EBS would be especially useful.

### Skin Samples

Skin samples were obtained for diagnostic purposes. After written informed consent, primary keratinocytes were isolated from the remaining tissue. Immortalized human control (NHK, normal human keratinocytes) and EBS keratinocytes (K14.R125C, K14.R125G, K14.N123S, K14.Y415H, K5.G138E/L175F and K5.E477D) were generated (Has et al. (2018) The Position of Targeted Next-generation Sequencing in Epidermolysis Bullosa Diagnosis. Acta Dermato-Venereologica 98:437-40; He Y, Maier K, Leppert J, Hausser I, Schwieger-Briel A, Weibel L, et al. (2016) Monoallelic Mutations in the Translation Initiation Codon of KLHL24 Cause Skin Fragility. Am J Hum Genet 99:1395-404). Keratinocytes were grown in low calcium Gibco™ Keratinocyte-SFM Medium supplemented with L-glutamine, EGF and BPE (LCM, Thermo Fisher Scientific) and cultured at 37° C., 5% CO₂ and 90% humidity. To induce cell-cell contacts, LCM was changed to high calcium medium (HCM, 1.2 mM CaCl₂) and cells were incubated for 24 h. For PKC412 treatment, NHK, K14.R125C or K14.R125G keratinocytes were seeded in medium without treatment. Next day, cells were washed twice with PBS, the medium was changed to fresh medium supplemented with 1 μM PKC412 (Biomol) or vehicle-control (DMSO) and incubated for 24 h. For the impedance spectroscopy measurements, PKC412 concentrations from 0.01-10 μM were tested.

### Heat-Shock Assay

To examine the behavior of the keratin cytoskeleton under a stress condition, the response after exposure to elevated temperature (“heat shock”) was examined. Cells were seeded onto glass cover slips in a 24-well plate and cultured at 37° C., 5% CO₂ and 90% humidity. Next day, the temperature was elevated to 42° C. for 15 min (in a water bath), and cells were fixed directly after the heat shock. To analyze the recovery of the keratin cytoskeleton, depleted medium was changed to fresh pre-warmed medium (37° C.), followed by fixing of the cells 30 min, 1 h, 2 h and 3 h after heat stress, then immunostaining using an antibody to K5.

### Epithelial Sheet Assay (Dispase Assay)

Cells were seeded in a 6-well plate, grown to confluency and then switched to HCM supplemented with 1 μM PKC412 or vehicle control for 24 h. Prior to the assay, cells were washed twice with 1.2 mM Ca²⁺ PBS and incubated for 30 min at 37° C. with Dispase II (Roche Diagnostics, 9 mg/ml in 1:2 HEPES buffer:keratinocytes medium, 2 mM Ca²⁺). Free-floating cell sheets were transferred to a 15 ml Falcon tube containing 5 ml 1.2 mM Ca²⁺ PBS and subjected to 25 rpm overhead rotation (WiseMix overhead rotator, Witeg Labortechnik, Germany) for 1 min at room temperature. Image acquisition was done using Nikon SMZ 1500 binocular and image processing by ImageJ.

### Phosphorylation Prediction of K14

Changes in K14 phosphorylation due to mutations leading to EBS were predicted using two tools: a) MIMP (Mutation IMpact on Phosphorylation), a machine-learning algorithm developed originally for cancer networking analysis, and b) MusiteDeep, a deep-learning algorithm that takes raw sequence data as input and uses convolutional neural networks to predict mutational impacts. MIMP works by constructing specificity models for kinases, which are then used to score phosphosites containing a mutation before and after the mutation to predict the impact it may have on phosphorylation. The training data for these algorithms were acquired from PhosphoELM, PhosphoSite Plus, HPRD, and PhosphoNetwork. A consensus score was created to minimize false positives. Using MusiteDeep, native and mutant protein phosphorylation predictions were determined separately for each kinase (CDK, PKC, PKA, MAPK, CK2, and other encompassing all other trained kinases) via input of an amino acid sequence. The resulting predicted phosphorylation scores (represented as a probability between 0 and 1) were then compared between specific mutant sequence and the native sequence, and an overall likelihood of change (loss or gain) was determined via a z-score calculation (difference between mutant and native likelihood of phosphorylation divided by the standard deviation of phosphorylation scores in the native sequence). A |Z-score|≥1 was determined to be a probable change in phosphorylation status, as long as either the mutant or native absolute score was ≥0.5 (threshold indicating that there is probable phosphorylation). As an example, if the standard deviation of a native sequence MusiteDeep score is 0.25, only a change of ≥0.25 with at least one of the mutant or native values ≥0.5 overall would yield a predicted change in phosphorylation.

### Immunofluorescence Analysis and Image Processing

Human keratinocytes grown on coverslips were fixed for 5 min in methanol (−20° C.) and 30 sec in acetone (−20° C.). Primary antibodies were diluted in 1% (w/v) BSA in TBS and incubated overnight at 4° C. in a humid chamber. The next day, coverslips were washed in TBS and incubated with the fluorophore-conjugated secondary antibody for 30 min at RT in the dark. DNA was stained with DAPI. Finally, coverslips were washed three times in TBS, rinsed briefly in aqua bidest and mounted with ProLong® Gold antifade reagent (Invitrogen). Images or Z-stacks were acquired with a confocal LSM 780 (Carl Zeiss) equipped with 40×/1.3 NA or 63×/1.4 NA oil immersion objectives and an AxioCam Mrm (Carl Zeiss). Image analysis and maximum intensity projections of Z-stacks was performed using Zen 2012 Blue software (Carl Zeiss). ImageJ was used for image processing.

### SDS-PAGE and Immunoblot Analysis

To separate proteins under denaturing conditions, sodium dodecyl sulfate polyacrylamide gel electrophoresis (SDS-PAGE) was performed. Proteins were transferred to nitrocellulose membranes (VWR) using a semi-dry blotter (Armin Baack Labortechnik). Membranes were blocked using 3% (w/v) skim milk/TBS with Tween (TBST) or 3% (w/v) bovine serum albumin (BSA)/TBST and subsequently probed with the appropriate antibodies. Chemiluminescence was detected using ChemoCam Imager (Intas). Band intensity was quantified using ImageJ software.

### Electrochemical Impedance Spectroscopy

Impedimetric measurements on NHK and K14.R125C keratinocytes were performed as previously described. Briefly, 8×10⁴ keratinocytes were seeded in 250 μl complete growth medium per well on self-developed multielectrode arrays (MEAs) with interdigital gold electrodes and cultivated for 2 to 3 days until cell layer confluence. Impedance spectra (500 Hz-5 MHz) were automatically recorded every 30 min using the impedance measurement platform based on an Agilent 4294A high-precision impedance analyzer (Agilent Technologies) and the self-developed controlling software IMAT advanced. After 2 to 3 days of pre-monitoring, the experiment was started by the application of PKC412 in high calcium medium (1.2 mM Ca²⁺) at the described concentrations. For control groups, the appropriate solvent was used. The cell signal (relative impedance) was determined with the self-developed analysis software IDAT v3.7.2.

### Phospho-Proteome Analysis

To perform phospho-proteome analysis, EBS K14.R125C keratinocytes were seeded in quintuples onto 10 cm dishes (3.5×10⁵ cells/ml). Next day, cells were treated with 0.8 μM PKC412 or DMSO in 1.2 mM Ca²⁺ medium and incubated for additional 24 h. Next day, cells were washed twice in PBS and lysed in 8 M Urea/50 mM TEAB buffer (500 μl per two 10 cm dishes). To degrade chromatin, cell lysates were sonified (10 min, cycle 30/30 sec), centrifuged for 15 min at 20,000×g and supernatants were transferred to new 1.5 ml tubes. For mass spectrometry analysis, lysates were reduced with DTT, alkylated with IAA and digested using LysC and trypsin in solution. Digested peptides were loaded on C18 double layer Stage Tips for further analysis. Phosphopeptides were enriched using the High-Select Tio2 Phosphopeptide enrichment kit (Thermo Scientific #A32993). Samples were analyzed by the Proteomics Facility at CECAD on a Q Exactive Plus Orbitrap mass spectrometer that was coupled to an EASY nLC (both Thermo Scientific). Peptides were loaded with solvent A (0.1% formic acid in water) onto an in-house packed analytical column (50 cm-75 μm I.D., filled with 2.7 μm Poroshell EC120 C18, Agilent). Peptides were chromatographically separated at a constant flow rate of 250 nL/min using the following gradient: 3-5% solvent B (0.1% formic acid in 80% acetonitrile) within 1.0 min, 5-30% solvent B within 121.0 min, 30-40% solvent B within 19.0 min, 40-95% solvent B within 1.0 min, followed by washing and column equilibration. The mass spectrometer was operated in data-dependent acquisition mode. The MS1 survey scan was acquired from 300-1750 m/z at a resolution of 70,000. The top 10 most abundant peptides were isolated within a 1.8 Th window and subjected to HCD fragmentation at a normalized collision energy of 27%. The AGC target was set to 5e5 charges, allowing a maximum injection time of 55 ms. Product ions were detected in the Orbitrap at a resolution of 17,500. Precursors were dynamically excluded for 25.0 s. All mass spectrometric raw data were processed with Maxquant (version 1.5.3.8) using default parameters. Briefly, MS2 spectra were searched against the canonical Uniprot Human FASTA (reference UP000005640, downloaded at 26.08.2020) database, including a list of common contaminants. False discovery rates on protein and PSM level were estimated by the target-decoy approach to 1% (Protein FDR) and 1% (PSM FDR) respectively. The minimal peptide length was set to 7 amino acids and carbamidomethylation at cysteine residues was considered as a fixed modification. Oxidation (M), Phospho (STY), and Acetyl (Protein N-term) were included as variable modifications. The match-between runs option was enabled. LFQ quantification was enabled using default settings. Further data handling was done in Perseus (version 1.6.15.0).

### Statistics

All statistics were processed using GraphPad Prism software, version 9.

TABLE S1
Summary of keratin filament organization of the
EBS-S cell lines examined under normal culture
conditions (37° C.) and heat stress (42° C.).

| Cell line | Phenotype at 37° C. | Phenotype at 42° C. |
|---|---|---|
| NHK | Keratin filaments | Bundled keratin filaments |
| K5.G138E/ | Bundled keratin filaments | Bundled keratin filaments ↑, |
| L175F | | keratin aggregates |
| K5.E477D | Short keratin filaments | Keratin aggregates |
| | at cell periphery | |
| K14.N123S | Perinuclear keratin bundles | Perinuclear keratin bundles ↑ |
| K14.R125C | Keratin aggregates | Keratin aggregates ↑↑ |
| K14.R125G | Keratin aggregates | Keratin aggregates ↑ |
| K14.Y415H | Altered keratin filaments | Keratin aggregates |

TABLE S2
List of putative kinase phosphorylation sites that were predicted
to be gained or lost using the computational tool MusiteDeep,
sorted by K14 mutation (p-site, phospho-site).

| | | Loss | Gain | |
|---|---|---|---|---|
| K14 | | possible | possible | Phosphorylation |
| Mutation | Domain | p-site | p-site | change |
| A94T | Head | — | — | no change |
| K116E | 1A | — | — | |
| K116N | 1A | — | — | |
| M119I | 1A | — | — | |
| M119T | 1A | — | — | |
| M119V | 1A | — | — | |
| Q120P | 1A | — | — | |
| Q120R | 1A | — | — | |
| L122F | 1A | — | — | |
| N123K | 1A | — | — | |
| N123S | 1A | — | — | |
| R125C | 1A | 128 | — | loss |
| R125G | 1A | 128 | — | |
| R125H | 1A | 128 | — | |
| R125L | 1A | 128 | — | |
| R125P | 1A | 128 | — | |
| R125S | 1A | — | 125 | gain |
| S128P | 1A | 128 | — | loss |
| Y129C | 1A | 128 | — | |
| Y129D | 1A | — | — | no change |
| L130P | 1A | — | — | |
| V133A | 1A | — | — | |
| V133L | 1A | — | — | |
| V133M | 1A | — | — | |
| R134C | 1A | — | — | |
| R134P | 1A | — | — | |
| L136P | 1A | — | — | |
| L136Q | 1A | — | — | |
| N140S | 1A | — | 140 | gain |
| L143P | 1A | — | — | no change |
| E144A | 1A | — | — | |
| R148C | 1A | — | — | |
| R211P | 1B | 213 | — | loss |
| V268D | L12 | — | — | no change |
| V270A | L12 | — | — | |
| V270M | L12 | — | — | |
| M272R | L12 | — | — | |
| M272T | L12 | — | — | |
| D273G | L12 | — | — | |
| A274D | L12 | — | — | |
| R288L | 2A | — | — | |
| M294T | 2A | — | — | |
| T319P | 2B | — | — | |
| I377N | 2B | — | — | |
| I377T | 2B | — | — | |
| E381K | 2B | — | — | |
| L384P | 2B | — | — | |
| R388C | 2B | — | — | |
| R388G | 2B | — | — | |
| R388H | 2B | — | — | |
| R388P | 2B | — | — | |
| L401P | 2B | — | — | |
| L408M | 2B | — | — | |
| E411K | 2B | — | — | |
| I412F | 2B | — | — | |
| I412N | 2B | — | — | |
| A413P | 2B | — | — | |
| A413T | 2B | — | — | |
| Y415C | 2B | — | — | |
| Y415H | 2B | — | — | |
| Y415S | 2B | — | — | |
| R416P | 2B | — | — | |
| R417P | 2B | — | — | |
| L418Q | 2B | — | — | |
| L418V | 2B | — | — | |
| L419Q | 2B | — | — | |
| E422K | 2B | — | — | |

TABLE S3
List of K14 putative kinase phosphorylation sites
that were predicted to be gained or lost using the
computational tools MIMP, sorted by K14 mutation.

| K14 | | Possible | Possible | Phosphorylation |
|---|---|---|---|---|
| Mutation | Domain | P-site | kinase(s) (family) | change |
| K116E | 1A | 118 | PLK | gain |
| Q120R | 1A | 118 | PKCα, PKCη | |
| E381K | 2B | 379 | PKCα | |
| L401P | 2B | 398 | Abl | |
| E411K | 2B | 414 | STE20: PAKA, | |
| | | | CAMKL: CHK1 | |
| L418V | 2B | 415 | Abl | |
| K116E | 1A | 114 | PKCα | loss |
| K116E | 1A | 118 | Aur | |
| K116N | 1A | 114 | PKCα | |
| K116N | 1A | 118 | Aur | |
| R125C | 1A | 128 | Akt, CAMK1/2, | |
| | | | CAMKL: AMPK/ | |
| | | | CHK1, DAPK, | |
| | | | MAPKAPK, PIM, | |
| | | | PKA, PKCδ/η, | |
| | | | PKD, RAD53, | |
| | | | RSK p70, SGK | |
| R125G | 1A | 128 | Akt, CAMK1/2, | |
| | | | CAMKL: AMPK/ | |
| | | | CHK1, DAPK, | |
| | | | MAPKAPK, PIM, | |
| | | | PKA, PKCδ, | |
| | | | PKD, RAD53, | |
| | | | RSK p70, SGK | |
| R125H | 1A | 128 | Akt, CAMK1/2, | |
| | | | CAMKL: AMPK/ | |
| | | | CHK1, DAPK, | |
| | | | MAPKAPK, PIM, | |
| | | | PKA, PKCδ, | |
| | | | PKD, RAD53, | |
| | | | RSK p70, SGK | |
| R125L | 1A | 128 | Akt, CAMK1/2, | |
| | | | CAMKL: AMPK/ | |
| | | | CHK1, DAPK, | |
| | | | MAPKAPK, PIM, | |
| | | | PKA, PKCδ, | |
| | | | PKD, RAD53, | |
| | | | RSK p70, SGK | |
| R125P | 1A | 128 | Akt, CAMK1/2, | |
| | | | CAMKL: AMPK/ | |
| | | | CHK1, DAPK, | |
| | | | MAPKAPK, PIM, | |
| | | | PKA, PKCδ/η, | |
| | | | PKD, RAD53, | |
| | | | RSK p70, SGK | |
| R125S | 1A | 128 | Akt, CAMK1/2, | |
| | | | CAMKL: AMPK/ | |
| | | | CHK1, DAPK, | |
| | | | MAPKAPK, PIM, | |
| | | | PKA, PKCδ, | |
| | | | PKD, RAD53, | |
| | | | RSK p70, SGK | |
| R211P | 1A | 213 | Aur, DMPK: ROCK, | |
| | | | PKA, PKCδ | |
| R416P | 2B | 414 | PKCα | |
| L418Q | 2B | 415 | Src: SrcB | |

TABLE S4
Enrichment analysis for most significantly up- and downregulated
gene ontology biological pathway (GOBP) categories.

| | | | |
|---|---|---|---|
| | | | Benj. |
| | Enrichment | | Hoch. |
| GOBP Category | factor | P value | FDR |

Fisher Exact Test: Most significantly upregulated

| | | | |
|---|---|---|---|
| cell junction organization | 1.4452 | 1.21E−60 | 1.70E−56 |
| cell junction assembly | 1.4391 | 4.15E−53 | 2.91E−49 |
| hemidesmosome assembly | 1.6848 | 2.30E−49 | 1.07E−45 |
| cytoskeleton organization | 1.2346 | 1.71E−44 | 5.98E−41 |
| cellular component disassembly | 1.4935 | 5.44E−42 | 1.53E−38 |
| involved in apoptosis | | | |
| hemidesmosome assembly | 1.562 | 1.53E−39 | 3.07E−36 |
| epidermis development | 1.495 | 2.42E−36 | 4.24E−33 |
| establishment or maintenance | 1.4138 | 8.92E−36 | 1.39E−32 |
| of cell polarity | | | |
| cellular component disassembly | 1.4999 | 1.47E−33 | 1.87E−30 |
| involved in apoptosis | | | |
| establishment of cell polarity | 1.5066 | 2.24E−33 | 2.62E−30 |

Fisher Exact Test: Most significantly downregulated

| | | | |
|---|---|---|---|
| ncRNA metabolic process | 0.4216 | 9.11E−40 | 2.13E−36 |
| ncRNA processing | 0.33824 | 4.86E−35 | 6.82E−32 |
| rRNA metabolic process | 0.27061 | 5.53E−33 | 5.17E−30 |
| primary metabolic process | 0.92422 | 1.11E−29 | 7.06E−27 |
| rRNA processing | 0.2889 | 2.00E−29 | 1.22E−26 |
| nitrogen compound metabolic | 0.90261 | 1.92E−28 | 1.08E−25 |
| process | | | |
| chromatin organization | 0.71586 | 3.53E−28 | 1.90E−25 |
| cellular nitrogen compound | 0.90317 | 8.00E−28 | 4.01E−25 |
| metabolic process | | | |
| cellular macromolecule | 0.92222 | 1.14E−27 | 5.50E−25 |
| metabolic process | | | |
| cellular macromolecule | 0.68249 | 7.21E−27 | 3.26E−24 |
| catabolic process | | | |

TABLE S5
Enrichment analysis for most significantly
up- and downregulated motif categories.

| | | | |
|---|---|---|---|
| | | | Benj. |
| | Enrichment | | Hoch. |
| Motif Category | factor | P value | FDR |

Fisher Exact Test: Most significantly upregulated

| | | | |
|---|---|---|---|
| Calmodulin-dependent protein | 1.15 | 1.38E−27 | 6.42E−26 |
| kinase II substrate motif | | | |
| PKC kinase substrate motif | 1.0834 | 5.91E−23 | 2.20E−21 |
| 14-3-3 domain binding motif | 1.1411 | 5.22E−21 | 1.62E−19 |
| PKA kinase substrate motif | 1.0677 | 1.08E−17 | 2.88E−16 |
| Akt kinase substrate motif | 1.2319 | 1.09E−13 | 2.53E−12 |
| MAPKAPK1 kinase substrate | 1.1949 | 3.75E−11 | 7.76E−10 |
| motif | | | |
| GSK3 kinase substrate motif | 1.1051 | 4.43E−11 | 8.23E−10 |
| Calmodulin-dependent protein | 1.1799 | 1.44E−09 | 2.06E−08 |
| kinase IV substrate motif | | | |
| Chk1 kinase substrate motif | 1.1675 | 1.41E−09 | 2.06E−08 |
| Plk1 PBD domain binding motif | 1.1103 | 1.92E−09 | 2.38E−08 |

Fisher Exact Test: Most significantly downregulated

| | | | |
|---|---|---|---|
| ERK1/2 kinase substrate motif | 0.87272 | 1.19E−46 | 2.20E−44 |
| GSK-3, ERK1, ERK2, CDK5 | 0.89122 | 7.92E−42 | 4.91E−40 |
| substrate motif | | | |
| WW domain binding motif | 0.89122 | 7.92E−42 | 4.91E−40 |
| β-Adrenergic Receptor kinase | 0.89218 | 1.34E−09 | 2.06E−08 |
| substrate motif | | | |
| CDK kinase substrate motif | 0.84721 | 4.05E−09 | 4.18E−08 |
| BARD1 BRCT domain binding | 0.75408 | 4.73E−09 | 4.63E−08 |
| motif | | | |
| Casein kinase I substrate | 0.90402 | 2.59E−08 | 2.41E−07 |
| motif | | | |
| Casein kinase I substrate | 0.89642 | 3.03E−08 | 2.69E−07 |
| motif | | | |
| BARD1 BRCT domain binding | 0.76834 | 2.19E−07 | 1.70E−06 |
| motif | | | |
| Growth associated histone | 0.9067 | 4.22E−07 | 3.14E−06 |
| HI kinase substrate motif | | | |

TABLE S6
List of antibodies including dilutions used in immunoblot
(IB) analysis and immunofluorescence (IF) staining.

| | | | IB | IF |
|---|---|---|---|---|
| | Source | Host | dilution | dilution |
| Primary antibody | | | | |
| K5 | Magin lab | rabbit | | 1:100 |
| Phospho-AKT substrate | Cell Signaling | rabbit | 1:500 | |
| RXXS*/T*) clone | | | | |
| 110B7E | | | | |
| P-K17 S44 | Cell Signaling | rabbit | 1:2,000 | |
| K17 | Magin lab | rabbit | 1:10,000 | |
| P-DSP S165/S166 | Cell Signaling | rabbit | 1:2,000 | |
| P-DSP S2849 | Magin lab | rabbit | 1:2,000 | |
| DSP1/2 | Magin lab | mouse | | 1:500 |
| DSP1/2 | Hatzfeld lab | rabbit | 1:10,000 | |
| α-Tubulin | Sigma | mouse | 1:10,000 | |
| Secondary antibody | | | | |
| anti-rabbit-Cy3 | Dianova | Donkey | | 1:800 |
| anti-mouse-A488 | Dianova | Donkey | | 1:800 |
| anti-mouse-, anti-rabbit | Dianova | Donkey | 1:20,000 | |
| HRP | | | | |

### Example 2. a PKC412 Dermal Delivery Formulation

This Example provides one example of a dermal delivery formulation. Also, see Franyoto et al., Journal of Physics: Conference Series 2019, 1217, 012151.

Powdered PKC412 can be homogenized with components #1-7 at 70° C. until uniform mixing is achieved. Warmed (70° C.) deionized water can be added over 10 min with continuous stirring and the mixture is cooled slowly to room temperature to form a cream. This formulation uses a concentration of PKC412 of 0.5%, but the concentration of PKC412 can range, e.g., from 0.01 to 5%. The concentration of Vaseline can range from about 2-25%. The concentration of stearic acid can range from about from about 3-30%. The concentration of sodium tetraborate can range from about 0.1-0.5%. The triethylamine concentration can range from about 0.5 to 3%. The concentration of propylene glycol can range from about 5 to 15%. The concentration of methylparaben can range from about 0.05 to 0.15%. Distilled water can be used for completing the mixture to 100%. Other fatty acids may be used to replace stearic acids.

| # | Components | Amount (g) | % |
|---|---|---|---|
| 1 | powdered PKC412 | 0.10 | 0.50% |
| 2 | Vaseline Album | 2.00 | 9.92% |
| 3 | Stearic acid | 3.00 | 14.87% |
| 4 | Na. Tetraborate | 0.05 | 0.25% |
| 5 | Triethylamine | 0.30 | 1.49% |
| 6 | Propylene glycol | 1.60 | 7.93% |
| 7 | Methylparaben | 0.02 | 0.10% |
| 8 | Distilled water | 13.10 | 64.95% |
| | Total | 20 | 100.00% |

### Example 3. a PKC412 Dermal Delivery Formulation

This Example provides one example of a dermal delivery formulation. Also, see Javadzadeh et al., Transcutol® (Diethylene Glycol Monoethyl Ether): A Potential Penetration Enhancer. In Percutaneous Penetration Enhancers Chemical Methods in Penetration Enhancement: Modification of the Stratum Corneum, Dragicevic, N.; Maibach, H. I., Eds. Springer Berlin Heidelberg: Berlin, Heidelberg, 2015; pp 195-205.

Powdered PKC412 can be homogenized in transcutol at 50° C. and then excipial hydrocream slowly added with stirring. The cream can be slowly cooled to room temperature with mixing. The formulation has a concentration of PKC412 of 0.1% but the concentration can range from about 0.01 to 5%. The concentration of Transcutol can range from about 3-15%. Excipial hydrocream can be used for completing the mixture to 100%

| # | Components | Amount (g) | % |
|---|---|---|---|
| 1 | powdered PKC412 | 0.03 | 0.10% |
| 2 | Transcutol | 1.5 | 4.76% |
| 3 | Excipial Hydrocream | 30 | 95.15% |
| | Total | 31.53 | 100.00% |

### Example 4. a PKC412 Dermal Delivery Formulation

This Example provides one example of a dermal delivery formulation. Also, see U.S. Pat. No. 4,164,564.

Powdered PKC412 can be homogenized with components #1-4 at 50° C. until uniform mixing is achieved. Warmed (50° C.) distilled water can be added over 10 min with continuous stirring and the mixture can be cooled slowly to room temperature to form a cream.

This formulation uses a concentration of PKC412 of is 0.24%, but the concentration of PKC412 can range from about 0.01 to 5%. The concentration of methylparaben can range from about 0.05 to 0.15%. The concentration of triglyceryl diisostearate can range from about 1-7%. The concentration of plastibase can range from about 3-32%. Distilled water can be used for completing the mixture to 100%.

| # | Components | Amount (g) | % |
|---|---|---|---|
| 1 | powdered PKC412 | 0.03 | 0.24% |
| 2 | methylparaben | 0.02 | 0.16% |
| 3 | triglyceryl diisostearate | 0.4 | 3.21% |
| 4 | Plastibase 50W | 2 | 16.06% |
| 5 | distilled water | 10 | 80.32% |
| | Total | 12.45 | 100.00% |

### Example 5. a PKC412 Dermal Delivery Formulation

This Example provides one example of a dermal delivery formulation. Also, see WO2004112794.

Powdered PKC412 can be homogenized with components #1-4 at 50° C. until uniform mixing is achieved. Warmed (50° C.) polyethylene glycol can be added followed by Cremophor over 10 min with continuous stirring, and the mixture can be cooled slowly to room temperature to form a cream.

This formulation uses a concentration of PKC412 of 0.11%, but the concentration of PKC412 can range from about 0.01 to 5%. The concentration of absolute ethanol can range from about 3 to 20%. The concentration of corn oil glycerides can range from about 10 to 40%. The concentration of alpha-tocopherol can range from about 0.05 to 0.3%. The concentration of polyethylene glycol can range from about 5-26%. Cremophor can be used for completing the mixture to 100%.

| # | Components | Amount (g) | % |
|---|---|---|---|
| 1 | powdered PKC412 | 0.05 | 0.11% |
| 2 | ethanol absolute | 3.39 | 7.21% |
| 3 | corn oil glycerides | 9.00 | 19.15% |
| 4 | DL alpha tocopherol | 0.05 | 0.11% |
| 5 | polyethylene glycol 400 | 13.00 | 27.67% |
| 6 | Cremophor RH 40 | 21.50 | 45.75% |
| | Total | 47 | 100.00% |

### Example 6. PKC412 Dermal Delivery Formulation

Additional formulations may be used to help with the delivery of the PKC412 through the skin. For example, colloidal systems that are typically lipid-based and include nanoemulsions, liposomes or flexible vesicles, nanostructured lipid carriers solid lipid nanoparticles (SLN) can be used. These nanosystems may improve bioavailability and efficacy, target delivery to skin regions and follicles, increase the stability of active, and facilitate the formulation of lipophilic, poorly water-soluble compounds. (Verma, et al., Nanomedicine: Nanotechnology, Biology and Medicine 2012, 8, 489-496; Zhang et al., ACS Applied Materials & Interfaces 2019, 11, 3704-3714.

The following abbreviations are made throughout the Examples. AML, acute myeloid leukemia; BSA, bovine serum albumin; CDK5, cyclin-dependent kinase 5; EBS, epidermolysis bullosa simplex; ERK, extracellular-signal regulated kinase; DMSO, dimethyl sulfoxide; DSP, desmoplakin; GO, gene ontology; GSK-3, glycogen synthase kinase 3; HCM, high calcium medium; IL, interleukin; INK, Jun N-terminal kinase; K, keratin; KF, keratin filament; LCM, low calcium medium; MAPK, mitogen-activated protein kinase; MEA, multi-electrode array; MMIP, mutation impact on phosphorylation; mTORC1, mammalian target of rapamycin complex 1; NHK; normal human keratinocytes; NM-IIA, non-muscle myosin heavy chain IIA; P, phosphorylation; PBS, phosphate buffered saline; PKA/C, protein kinase A/C; PTM, posttranslational modification; RSK, p90 ribosomal S6 kinase; S6K, p⁷⁰ ribosomal protein S6 kinase; SDS-PAGE, sodium dodecyl sulfate polyacrylamide gel electrophoresis; SM, advanced systemic mastocytosis.

### Example 7. PP2 Protects from Keratin Mutation-Associated Liver Injury and Filament Disruption Via SRC Kinase Inhibition

The results described herein highlight the significance of several findings: (i) Targeting of SRC tyrosine kinase, using the inhibitor PP2, offers a new therapeutic approach for IF-associated diseases as demonstrated by findings in K18 R90C-expressing A549 cells and K18-R90C transgenic mice; (ii) PP2 and vemurafenib preferentially protect livers of male but not female mice from Fas-mediated injury, as contrasted with PKC412 which provided similar protection in both sexes; (iii) The mechanism of PP2 action involves SRC kinase inhibition, but SRC kinase activity itself appears to be required for the PP2 effect; and (iv) PP2 is rapidly metabolized in mice, particularly in females when compared to males which explains its sexual dimorphic effect. It should be noted that, even though the findings described herein in mice show a male-selective effect, metabolism in humans may be different than in mice. Hence, it is contemplated that these treatments may be effective for both male and female humans.

Because changes in K8 and K18 phosphorylation typically occur at Ser/Thr instead of Tyr residues during cellular stress, the identification of the SRC tyrosine kinase inhibitor PP2 as a drug that normalizes keratin mutation-induced filament disruption is somewhat unexpected. For example, the highly abundant keratins [K8/K18 make up 0.3% of total cellular proteins in mouse liver] become hyperphosphorylated and serve as a Ser/Thr ‘phosphate sponge’ during cellular stress by shunting away the phosphorylation and subsequent activation of pro-apoptotic targets, thereby protecting from hepatocyte injury. Given the lack of detectable K8/K18 Tyr phosphorylation changes in response to PP2 as compared with K8/K18 Ser/Thr phosphorylation, it was positted that PP2's global effect on reducing K8/K18 phosphorylation is achieved primarily through a Ser/Thr kinase intermediate, with RAF kinase being a likely candidate. This is based on the findings that the RAF kinase inhibitor vemurafenib also provided a similar protective effect to PP2 from Fas-mediated liver and cell injury.

The normalization by PP2 of mutation-triggered keratin cytoskeletal disruption and protection from apoptosis, lends further support for targeting the perturbed keratin phosphorylation accompanied by stress-induced activation of kinases (be it environmental or genetic stress) as a potential therapeutic approach. Another different small molecule is PKC412 (also called Midostaurin), a Ser/Thr kinase inhibitor and FDA-approved drug used to treat acute myeloid leukemia associated with FLT3 mutations and systemic mastocytosis, also ameliorates the negative effect of the K18 R90C mutation. The epidermal growth factor receptor inhibitor, afatinib, is another kinase inhibitor that corrects epidermal keratin 14 mutant aggregation in cultured cells. The use of kinase inhibitors has also been successful in protecting animals from cardiomyopathy caused by mutations in the nuclear IF lamin A/C. There are currently 68 FDA-approved therapeutic agents that target more than 20 different protein kinases and are primarily used to treat malignancies and a few inflammatory disorders. Examples of FDA-approved drugs that are SRC kinase selective inhibitors and could be tested for keratin mutation associated disorders include Bosutinib and Dasatinib. The different signaling pathways impacted by PP2 compared with PKC412 raise the possibility that combinations of kinase inhibitors may have an added protective benefit.

The SRC tyrosine kinase inhibitor PP2-mediated a dramatic decrease in K8/K18 Ser/Thr phosphorylation with limited change in keratin tyrosine phosphorylation. This, coupled with the findings in the A549 cell culture model and the K18 R90C mouse liver cytoprotection, suggest that PP2 acts on K8/K18 indirectly. The PP2 mode of action is different to that observed for PKC412 since the latter leads to hypophosphorylation of a keratin-stabilizing binding protein (NMHC-IIA) while PP2 leads to global desphosphorylation of K8/K18. The observed effect of PP2 is clearly SRC protein dependent and is likely to be mediated by regulating downstream SRC-kinase-related Ser/Thr kinases. These results indicate that one likely downstream kinase candidate is RAF given its known role as a K8/K18 kinase, and the phenocopying of the PP2 effect in cultured cells and K18 R90C mice by the RAF kinase inhibitor vemurafenib. The known activation of RAF [a K8 binding partner and K18 kinase] by SRC, and these finding that the RAF inhibitor vemurafenib phenocopies PP2 in terms of its hepatoprotective effects, lends support to the proposed PP2 mechanism summarized herein. However, it cannot be ruled out that PP2 has additional effects on other kinases and/or phosphatases.

These results also showed that PP2's hepatoprotective effect is restricted to male mice, likely due to the more rapid hepatic turnover of PP2 in female mice. Sexual dimorphism has been reported in mice treated with other tyrosine kinase inhibitors, including emodin and genistein. Sex-dependent differences in liver cytochrome P450 (CYP) content has been described, with female mice reportedly having total CYP content that was two-fold greater than that of male mice. Herein, PP2 selectively rescued K18 R90C mutation-induced filament disruption and susceptibility to Fas- and APAP-mediated liver injury in male mice.

In summary, these findings show that SRC and downstream RAF kinase inhibition protect against hepatocyte keratin mutation-triggered cytoskeletal disruption/aggregation and predisposition to apoptosis. From a clinical application perspective, consideration may be given for the potential use of select kinase inhibitors such as PKC412, PP2-like compounds, or vemurafenib in patients with keratin mutations for prophylactic and/or therapeutic applications. For example, prophylaxis or treatment (in the form of safe oral or cream formulations) interventions can be envisioned in patients with the blistering skin disease EBS who have filament disrupting mutations such as K14 R125C to potentially prevent blister formation or to treat blisters. Similarly, K8/K18 filament disrupting variants that are ‘silent’ under basal conditions but associate with fatal drug-induced liver injury may be candidates for similar interventions. This paradigm could extend to other IF-associated diseases as a potential therapeutic approach via repurposing of existing FDA-approved drugs or the use of novel compounds.

### Materials and Methods

### High-Throughput Drug Screening and Analysis

The screening strategy was performed as described. In brief, human A549 lung adenocarcinoma cells (American Type Culture Collection) were seeded into six-well plates overnight and then transduced with K8 WT and either the GFP-tagged K18 WT or GFP-tagged K18 R90C lentivirus for 2d. A549 cells were selected for the screening assay because of their high transduction efficiency and readily visualized keratin filaments. The transduced cells were seeded into 384-well plates for 1d followed by addition of compounds from the Navigator Pathways (Center for Chemical Genomics, University of Michigan). After 48 h, cells were fixed counterstained with 4′,6-diamidino-2-phenylindole (DAPI; Invitrogen). Images were taken with the Image Xpress Micro XLS High Content Imaging System then analyzed using MetaXpress software (Molecular Devices). Compounds that decrease the number of keratin dots/cell and promote keratin filament formation were selected for secondary validation. 945 compounds from the Navigator Pathways library were screened. The library consists of several daughter sets that cover a range of pathways including Autophagy, Wnt, Epigenetics, Protein Kinases, Proteases, Redox, Cannabinoids, and Natural Products. A total of 24 compounds reduced keratin mutation-induced filament collapse into dots upon initial screening, and this study was focused on PP2 (Selleckchem) given its inhibitory role on tyrosine phosphorylation which differs from prior findings with the Ser/Thr kinase inhibitor PKC412.

### Cell Culture and Apoptosis Induction

A549 cells were cultured using Ham's F-12K medium (Thermo Fisher Scientific) supplemented with 10% fetal bovine serum (Sigma Aldrich) and 1% penicillin and 10,000 U/mL streptomycin (Life Technologies). To test susceptibility to apoptosis, A549 cells were transduced with GFP-K18 WT or GFP-K18 R90C lentiviruses, followed by pretreatment with vehicle [dimethyl sulfoxide (DMSO)] or the drugs PP2 or vemurafenib (Selleckchem) (48 h) before addition of IFN-γ (R&D Systems; 40 ng/mL, 6 h) then FasL (CH11; Millipore; 100 ng/mL, 12 h).

### Animal Studies

All mouse experiments were performed using a protocol approved by Rutgers University Institutional Animal Care and Use Committee. Previously described 4-6 weeks-old F22 male and female mice that express the human K18 R90C mutant (FVB/N background) were used. Mice were treated daily with PP2 (1 mg/kg body weight) or DMSO for 4d by intraperitoneal injection. For immunofluorescence experiments, mouse livers were collected after CO₂ euthanasia and embedded in optimum-cutting-temperature compound then stored (−80° C.). For Fas experiments, mice were treated by intraperitoneal administration of Fas antibody (0.15 μg/g body weight; BD Pharmingen). After 5 h, mice were sacrificed followed by collection of blood and liver. Livers were fixed with 10% formalin and analyzed by hematoxylin-eosin (H&E) staining or TUNEL assay. A hemorrhage score was calculated using QuPath (Quantitative Pathology & Bioimage Analysis) and ImageJ software. Apoptosis was estimated using TUNEL assay detection kit (ApopTag Peroxidase In Situ Apoptosis; Sigma Aldrich). Fluorescence images were acquired with the same exposure time and used to count the apoptotic nuclei. Serum alanine aminotransferase (ALT) levels were determined using the Liquid ALT Reagent Set (Pointe Scientific). For the acetaminophen (APAP) experiments, the liver histopathology score was determined using a 0-4 scoring system. Scoring was conducted by two scorers blinded to treatment conditions, and the average score was used.

### Biochemical and Immunofluorescence Analysis

Cell and liver samples were solubilized using a Dounce homogenizer in 2% SDS-containing PBS supplemented with protease inhibitor (Thermo Fisher Scientific), then sonicated 80× (is pulse, intensity 70) (Fisher Scientific; Model: FB50). Lysates were pelleted (16,000 g×10 min/20° C.) and the supernatant was used for subsequent analysis. Equal amounts of protein were separated by SDS-polyacrylamide gel electrophoresis (PAGE) then transferred to polyvinylidene fluoride membranes (Bio-Rad) followed by immunoblotting. For immunoprecipitation, K18 R90C-transduced A549 cells were homogenized in PBS containing 1% Nonidet P-40 (NP40), protease inhibitors, and okadaic acid (Cayman Chemical; 1 μg/mL). After mixing (1 h/4° C.), lysates were pelleted (16,000 g×10 min/4° C.) followed by immunoprecipitation (overnight/4° C.) using anti-human K18 antibody conjugated to Protein A/G Magnetic Beads (Thermo Fisher Scientific) then analyzed by mass spectrometry or blotted with antibodies that selectively recognize phosphorylated K8 or K18. For mass spectrometry, conjugated beads were suspended in PBS-buffered 8M urea to yield ˜6 g (in 60 μl urea-containing buffer). Samples were separated SDS-PAGE, followed by in-gel tryptic digestion then LC-MS/MS analysis. Peptides were identified by the Mass Spectrometry Facility at Rutgers University. Immunofluorescence staining of cells and tissues was performed as described, and images were visualized using a ZEISS Axio Imager M2 microscope or Leica Thunder Imager.

Gene Silencing with Small Interfering RNA (siRNA)

SRC siRNA and PTK6 siRNA duplexes were obtained from Origene (Catalog #SR304574, Catalog #SR321510). Scrambled siRNA from Origene (Catalog #SR30004) was used as control. Transfections were conducted using the Lipofectamine RNAiMAX (Thermo Fisher Scientific) according to the manufacturer's protocol.

### PP2 Metabolism Analysis

PP2 stock solution was prepared in dimethyl acetamide to a final concentration of 3.3 mM. Male and female FVB mice (4-6 weeks old) were injected with PP2 (1 mg/kg body weight). Mice were sacrificed and livers were harvested at the indicated times. For PP2 extraction, 0.5 g liver tissue was homogenized [100 strokes using Dounce, in 2 mL 2% SDS-containing PBS (pH7.4)] then sonicated (twenty 2 sec pulses). Equal protein lysates (assayed by BCA method; Thermo Fisher Scientific) were aliquoted (200 μL fractions) in microcentrifuge tubes and 800 μL methanol was added/fraction to precipitate the protein followed by pelleting (20,230 g×10 min/20° C.). The supernatant was transferred to new tubes and pelleted to remove any remaining debris (20,230 g×10 min/20° C.). The supernatants were pooled into 50 mL conical tubes then diluted 1:1 (v:v) using 200 mM ammonium formate+4% phosphoric acid then pelleted (5,250 g×20 min/20° C.). The supernatant was then filtered using 0.2 m syringe filter (Waters Corporation, Part #WAT200504) and used for solid phase extraction (SPE) by loading onto Waters Oasis MCX SPE columns (Waters Corporation, Part #186000252). The column was washed once with 1 mL 100% methanol and eluted with 1 mL 5% NH₄OH in methanol. The eluant was pelleted (20,230 g×10 min/20° C.) and the supernatant was transferred to a glass tube and dried using a Speedvac vacuum concentrator (Thermo Fisher Scientific). The evaporated sample was resuspended in 100 L injection solvent [80% (5% ammonium hydroxide in methanol)+20% (10 mM ammonium formate+0.1% formic acid)], and 50 L was injected for ultra-performance liquid chromatography (UPLC). Serum proteins were precipitated by adding 400 L methanol to 100 L serum, then pelleted (20,230 g×10 min/20° C.). The supernatant was transferred to new tubes then processed further for SPE and UPLC exactly as carried out for the liver tissue extracts.

### UPLC Analysis of PP2

A Waters ACQUITY UPLC system equipped with Empower software and H-Class PLUS (CH-A) Core was used. A reverse-phase octadecylsilica (C18) ACQUITY UPLC BEH Shield RP18 Column (130 Å, 1.7 m, 3 mm×100 mm; Part #186004669) and ACQUITY VanGuard Pre-column (130 Å, 1.7 m, 2.1 mm×5 mm; Part #186003977) were used. Absorbance was recorded at 254 nm. The column was eluted at flow rate of 0.5 ml/min with linear gradients of solvents A and B (A, 10 mM ammonium formate, 0.1% formic acid in water; B, 0.1% formic acid in methanol). The solvent gradient was as follows: 0-1 min, 50-100% B; 1-3 min, 100-100% B; 3-3.5 min, 100-50% B; 3.5-10 min, 50-95% B.

### Limit of Detection (LOD) and Limit of Quantitation (LOQ) Calculation

PP2 standard curve concentration was created by plotting the area under the curve for the PP2 peak versus mass of PP2 injected. Standard curve regression analysis was performed to calculate the slopes and standard deviation of the intercept (σ). LOD [3.3*(σ/s)] and LOQ [10*(σ/s)] were then calculated.

### Statistical Analysis

Statistical analysis was performed using GraphPad Prism-7 software. Statistical comparisons were done using the unpaired Student's t-test or, for samples with three or more groups, by one-way ANOVA followed by the Tukey post-hoc test. Experimental data are expressed as the mean±standard deviation (SD).

### Results

### PP2 Reverses Keratin Aggregate Formation and Protects Against Apoptosis in K18 R90C-Expressing Cells

A549 cells that express K18 R90C were used in a drug-screening assay as described in Materials and Methods. The readout was conversion of keratin aggregates (i.e., dots as visualized by GFP fluorescence) to filaments. PP2, a SRC kinase inhibitor, significantly decreased the percentage of cells with keratin mutant aggregates at 5 μM. Normalization of the keratin organization led to protection of the K18 R90C but not wild-type K18-expressing A549 cells from apoptosis induced by interferon-7 (IFN-7) and Fas ligand (FasL). This conclusion is based on decreased presence of cleaved caspases 3 and 7, and a decrease in the TUNEL-positive cells from 37% to 8% upon PP2 treatment. PP2 appears to inhibit the growth of A549 cells, as previously reported in other cell lines, but does not have an effect on body or liver weight. Notably, the numbers of TUNEL-positive cells were comparable between PP2 and DMSO treatments under basal non-Fas condition, thereby indicating that at the administered dose (5 μM), PP2 does not induce apoptosis.

### PP2 Protects Against Liver Injury in Male but not Female K18 R90C Mice

K18 R90C, when expressed in transgenic mice, results in collapse of keratin filaments into dots and predisposition to Fas-induced liver injury. The efficacy of PP2 was evaluated in vivo by administering PP2 intraperitoneally for 4 consecutive days into mice expressing the human K18 R90C mutant. Administration of PP2 normalized keratin filaments in male but not female livers. In male but not female mice, PP2 improved parenchymal liver hemorrhage and ALT levels. This protection was also supported by resistance to apoptosis, with PP2 treatment leading to fewer TUNEL-positive cells. Similar trends were also noted after immunoblot analysis of cleaved caspases 3 and 7, and in the protection from degradation of keratins. As expected, PP2 decreased SRC phosphorylation at its stimulatory phospho-site Y416 in male but not female livers.

The previously reported liver injury protection by the Ser/Thr kinase inhibitor PKC412 had not been compared in male versus female mice. Direct comparison of male versus female mice showed that PKC412 equally protects both sexes from Fas-induced liver injury. Therefore, the Tyr-kinase inhibitor PP2 exerts its hepatoprotective effect in a male selective manner while the Ser/Thr kinase inhibitor PKC412 protects both sexes.

### SRC but not PTK6 Expression is Required for PP2-Mediated Filament Normalization

To test whether SRC plays a definitive role in PP2 action, the effect of SRC-knockdown on keratin filament organization was tested in GFP-K18 R90C lentivirus-transduced A549 cells. siSRC markedly reduced total SRC level without altering the expression of endogenous K8/K18 nor of exogenously introduced GFP-K18. Notably, SRC knockdown blocked the protective effect of PP2 but did not by itself phenocopy the PP2 protective effect. This suggests that SRC activity inhibition is a biologic effect functionally different than SRC knockdown. This also indicates that SRC kinase activity and potential dynamic changes are essential for PP2 action, and its complete removal abolishes PP2's ability to inhibit the kinase. Of note, PP2 has a dramatic inhibitory effect on SRC kinase activity in A549 cells, which is supported by its inhibitory effect of SRC-Y416 phosphorylation.

Importantly, another reported target for PP2 is the protein tyrosine kinase (PTK)6 (24). Therefore, the effect of PTK6 knockdown was tested as was done for SRC knockdown. In contrast to SRC knockdown, PTK6 knockdown did not abolish PP2's effect. Therefore, SRC activity is selective for PP2 action in that not all kinases known to be inhibited by PP2 contribute to keratin filament aggregation imparted by the K18 R90C mutation.

### PP2 Decreases Global Phosphorylation Levels of K18 and K8

Given the known effects of keratin and other intermediate filament protein phosphorylation on their cellular organization, and the increase in keratin phosphorylation caused by K18 R90C, the effect of PP2 on K8/K18 phosphorylation was tested. For this, keratins from PP2- and DMSO-treated K18 R90C-expressing cells were immunoprecipitated and assessed keratin phosphorylation by mass spectrometry. Notably, PP2 decreased global phosphorylation of K18 and K8 by 43% and 38%, respectively. Some of the changes observed by mass spectrometry were validated by immunoblot analysis including the PP2-mediated significant decrease in K18 and K8 at S34 and S432, respectively. Furthermore, the induced hyperphosphorylation of K18/K8 by the Ser/Thr phosphatase inhibitor, okadaic acid, was reversed by PP2 in A549 cells.

### Vemurafenib Protects Against Apoptosis in Cultured Cells and Male Mice

The mass spectrometry phosphorylation results showed that Ser/Thr but not Tyr phosphorylation was decreased by PP2, thereby implying that K18/K8 phosphorylation was not regulated directly by SRC. This led us to hypothesize the involvement of a Ser/Thr kinase intermediate that leads to hypophosphorylation of K8 and K18 by PP2. Since RAF is a direct Ser/Thr kinase downstream of SRC, and directly binds to K8 and phosphorylates K18, it was tested whether inhibition of RAF has similar protective effect against Fas-induced apoptosis. K18 R90C-expressing cells were treated with vemurafenib, a well-established and FDA-approved RAF kinase inhibitor. Vemurafenib protected cells from apoptosis in a dose-dependent manner by decreasing cleaved caspase 3 but not caspase 7 levels. TUNEL staining showed less nuclear punctae after vemurafenib treatment, thereby supporting the anti-apoptotic effect of vemurafenib in K18 R90C-expressing cells as well as the involvement of RAF kinase in PP2 action. To assess in vivo efficacy, vemurafenib was then tested in K18 R90C mice. Similar to the findings with PP2, pretreatment of vemurafenib at 1 mg/kg body weight protected male but not female K18 R90C mice from Fas-induced liver injury and caspase activation. Vemurafenib didn't effect mouse liver or body weight, but a higher vemurafenib dose (5 mg/kg body weight) appeared to be toxic.

Acetaminophen (APAP) was also tested as a therapeutic model, which is more feasible experimentally than the Fas model because the severity of injury of the Fas model in K18 R90C mice makes it challenging to test a therapeutic effect. K18 R90C mice were injected with APAP for 3 h then treated with PP2 or vehicle for 5 h. Although the primary mode of mouse liver injury in APAP is necrosis, apoptosis also takes place albeit to a lower extent than necrosis. PP2 led to a significant reduction in caspase 7 activation in males (compared with vehicle controls) and significant histologic protection when comparing male with female PP2-treated mice but there was no significant difference in the tested necrosis markers. This suggests that PP2 might preferentially protect from apoptosis rather than necrosis pathways, but other experimental liver injury models will require testing and validation.

### Sexual Dimorphism in Hepatic PP2 Metabolism

In order to define the sex dimorphic effect of PP2 in male versus female mice, an assay was developed to measure its levels in mouse liver and serum. PP2 is a heterocyclic amine with both polar and hydrophobic regions. Using UPLC-UV and a reverse-phase C18 column, PP2 standards were efficiently detected in picomole ranges with a limit of detection of 1.2 pmole, and a limit of quantitation of 3.6 pmole. In addition, there was no matrix interference such as serum proteins in the detection method.

PP2 (1 mg/kg body weight) was then administered to male and female FVB/N mice then assayed PP2 content in liver and serum. PP2 was detected in both male and female mouse livers 1 h after administration, while in serum there was no detectable PP2. Comparison of the UV-Visible absorbance of a PP2 standard with that of the eluting peak observed in liver samples confirmed that the compound detected in mouse liver was indeed PP2. The rate of hepatic clearance of PP2 is rapid in that after 4 h there was no detectable PP2 in male or female mice. However, PP2 levels as a function of time showed that male mouse liver had significantly higher PP2 levels compared to female mouse liver. This could be due to a difference in hepatic uptake or metabolism in male versus female mice, and likely explains the selective protective effect of PP2 in male but not female mice.

### Example 8

Epidermolysis bullosa simplex (EBS) is a blistering skin disorder caused by mutations in keratin 5 (K5) and keratin 14 (K14) genes. Normal K5/K14 form extended fibers in skin cells (keratinocytes) and provide critical mechanical integrity via keratin-keratin or keratin-nonkeratin protein interactions that are regulated by dynamic addition/removal of ion-charged modifications called phosphates. Severe EBS has no cure and results in severe skin blistering throughout the body starting at infancy. Upon mild stress, EBS-causing mutations lead to aggregation of keratin fibers in the cytoplasm with consequent cell rupture and replacement of dead cells by fluid (i.e., blisters). The two compounds are focused on herein, named PKC412 and vemurafenib, are currently being used as oral drugs to treat patients with cancer. It was hypothesized that PKC412 and vemurafenib can be repurposed as a cream and used alone or as a cocktail to treat patients, e.g., with severe EBS. A purpose of the experiments described herein are to study PKC412 and vemurafenib in keratinocyte cultures expressing two different K14/K5 mutations, determine whether PKC412+vemurafenib cocktail, at lower doses and predictably less toxicity, is more beneficial than individual compounds. Individual PKC412 or vemurafenib and cocktail creams will be tested on cadaver skin, skin explants and organotypic keratinocyte cultures, followed by using optimized dosing in EBS as part of a compassionate preclinical trial assessment to confirm their effectiveness in such treatment. As such, the use of PKC412, PP2-like drugs, and vemurafenib (Vem) as a potential therapeutics alone or in combination are contemplated.

Whether a cocktail treatment with PKC412 and vemurafenib is more beneficial in effectiveness than individual compound usage is described herein.

Preliminary Results: The hypothesis was tested that, in A549 epithelial cells and transgenic mice expressing K18 R90C, that combination exposure to two drugs at 0.5× dosing, as compared with the individual compound exposure at 1× each dosing will have at least a similar if not better protective effect, while also evaluating potential side effects. As shown herein, 0.5× dosing of PKC412+PP2 had a similar effect in protecting A549 cells, that express K18 R90C, from apoptosis, while 1×PKC412+1×PP2 also had a prominent protective effect. Importantly, doubling the dose of PP2 to 10 μM is not well tolerated in these cells thereby showing protection from toxicity when using the 1×+1× dosing, as was the case in terms of decreased inhibition of cell growth for the 0.5× dosing of the two drugs when compared with 1×.

The following were then tested in transgenic mice, the 0.5×+0.5× of PKC412+PP2 or PKC412+Vein. Notably, the PKC412+PP2 or PKC412+Vein half-dose cocktail treatment further improved parenchymal liver hemorrhage as compared with a single 1× treatment. Also, the increase in serum alanine aminotransferase (ALT) level (a marker of liver injury) after Fas treatment becomes significantly reduced upon PKC412, PP2 or Vein pretreatment. PKC412+Vein half-dose cocktail treatment further reduced serum ALT compared with PKC412 or Vein treatment alone. The significant difference in ALT levels (compared with the untreated group) upon Fas-L treatment diminished in the cocktail pretreatment groups.

Importantly, as a proof of principle, the effect of Vein alone or in combination with PKC412 in the human keratinocyte K14 R125C cell line was tested. Vein alone at concentrations of 1 μM and 2.5 μM had a significant effect in decreasing the number of keratinocytes with keratin aggregates. Of note, 1 μM of Vein in A549 cells did not have a significant effect in protecting the cells from apoptosis (they required 5 μM to observe an effect). In the experiment with K14 R125C cells, the keratinocytes appeared to be more sensitive to Vein than A549 cells. In depth analysis will be carried out for Vein alone in both the K14 R125C and K5 E477D cell lines and compared with varied doses of PKC412, then as a cocktail. Addition of one-half the concentration of PKC412 (0.4 μM) did not further decrease the number of keratinocytes with keratin aggregates.

One important experimental aspect of this Example is the testing of a range of combinatorial dosing to complement the 0.5×+0.5× dosing that is shown here for the K18 system as a proof of principle. The dose ranges that will be tested are summarized in the table below. The starting X dose will be derived with confidence after at least 3 consistent biologic replicates (aside from duplicate technical replicate for each biologic replicate). The dosing conditions listed in the table will be tested in: (i) the K14 R125C and K5 E477K keratinocyte cell lines, (ii) dispase assay, (iii) cell stretch assay, and (iv) organotypic cultures. The optimal combination will then be tested in human cadaver skin, organotypic keratinocyte cultures and skin explants. The optimal combination will also be tested in an established stretch assay to examine whether it improves mechanical stress resilience.

### Proposed Combination and Single Dosing Controls

| PKC | | | Vem | | |
|---|---|---|---|---|---|
| enriched | PKC412 | Vem | enriched | PKC412 | Vem |
| Condition 1P | 2x | None | Condition 1V | None | 2x |
| Condition 2P | 1x | None | Condition 2V | None | 1x |
| Condition 3P | 0.75x | 0.25x | Condition 3V | 0.25x | 0.75x |

| | | | |
|---|---|---|---|
| Condition | 0.5x | 0.5x | (Equal dosing of 0.5x each) |
| PV 0.5x | | | |

| | | | |
|---|---|---|---|
| (Equal dosing of 1x each) | Condition | 1x | 1x |
| | PV 1x | | |

Potential Advantages of the Combination Drug Approach: An exciting aspect of this discovery is that cocktail treatment with PKC412 and Vemurafenib may be more beneficial than individual compound usage in terms of effectiveness and/or toxicity profile. Indeed, the experiments in mice that express the K18 R90C mutation are highly encouraging in that a cocktail approach is likely to be beneficial. The drug cocktail/combination approach is routinely used in a variety of diseases, particularly in cancers and infectious diseases; however, in the case of EBS this would likely be a new approach. Should the combination (PKC412+Vem), PKC412 and Vein administered alone, or one of the two drugs alone, protect both K5 and K14 mutant keratin expressing cells that lead to severe EBS, it would indicate that the test compound(s) will also be beneficial in patients with severe EBS who harbor other keratin mutations. In addition, should Vein behave similarly to PKC412 in providing protection from mutant keratin aggregation and mechanical stress (tested via the dispase assay or cell stretching), it will indicate that the cocktail may also be effective when tested as a formulation for topical administration, e.g., a cream or ointment formulation, in patients with EBS.

1. A formulation for topical administration that comprises a therapeutically effective amount of the compound PKC412 having the following structure 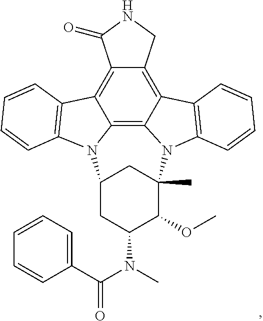 [SMILES: CO[C@@H]1[C@H](N(C)C(=O)c2ccccc2)C[C@H]2C[C@]1(C)n1c3ccccc3c3c4c(c5c6ccccc6n2c5c31)C(=O)NC4]
or a salt thereof.

2. The formulation of claim 1, which comprises a concentration of about 0.1% to 5% PKC412, or a salt thereof.

3. The formulation of claim 2, which comprises a concentration of about 0.5% PKC412, or a salt thereof.

4. The formulation of claim 1 that further comprises vemurafenib, or a salt thereof.

5-8. (canceled)

9. The formulation claim 1, wherein the formulation for topical administration is a cream or ointment.

10. A method to treat a human patient having epidermis bullosa simplex (EBS), epidermolytic hyperkeratosis, epidermolytic palmoplantar keratoderma, palmoplantar keratoderma, nonepidermolytic, pachyonychia congenita type 1, or pachyonychia congenita type 2, comprising administering to the patient a therapeutically effective amount of the compound PKC412 having the following structure 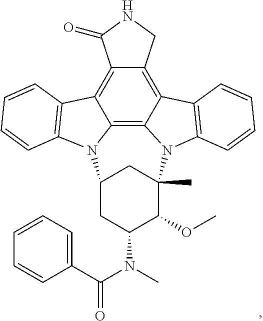 [SMILES: CO[C@@H]1[C@H](N(C)C(=O)c2ccccc2)C[C@H]2C[C@]1(C)n1c3ccccc3c3c4c(c5c6ccccc6n2c5c31)C(=O)NC4]
or a salt thereof.

11. The method of claim 10, wherein the PKC412, or salt thereof, is administered orally.

12. The method of claim 10, wherein the PKC412, or salt thereof, is administered topically to the patient's skin.

13. The method of claim 12, wherein the PKC412, or salt thereof, is administered topically to the patient's skin in a cream or ointment formulation.

14. The method of claim 13, wherein the cream or ointment formulation comprises a concentration of about 0.1% to 5% PKC412, or salt thereof.

15. The method of claim 13, wherein the cream or ointment formulation comprises a concentration of about 0.5% PKC412, or salt thereof.

16. The method of claim 13, wherein the formulation further comprises vemurafenib, or a salt thereof.

17. A method for treating a subject having or at risk for developing complications from an intermediate filament associated disease, comprising administering to the subject an effective amount of a SRC and/or RAF kinase inhibitor to treat the intermediate filament associated disease.

18. The method of claim 17, wherein the subject is a human male.

19. The method of claim 17, wherein the subject is a human female.

20. The method of claim 17, wherein the kinase inhibitor is PP2, or a salt thereof, or vemurafenib, or a salt thereof.

21. The method of claim 17, wherein the intermediate filament associated disease is epidermis bullosa simplex, epidermolytic hyperkeratosis, epidermolytic palmoplantar keratoderma, palmoplantar keratoderma, nonepidermolytic, pachyonychia congenita type 1, or pachyonychia congenita type 2.

22. The method of claim 17, comprising administering PKC412, or a salt thereof, in combination with the SRC and/or RAF kinase inhibitor.


SEQUENCE LISTING (8 sequences)

SEQ ID NO: 1 - protein, 36 aa, synthetic construct
EKVTMQNLNDRLASYLDKVRALEEANADLEVKIRDW

SEQ ID NO: 2 - protein, 36 aa, synthetic construct
EKVTMQNLNDCLASYLDKVRALEEANADLEVKIRDW

SEQ ID NO: 3 - protein, 590 aa, synthetic construct
MSRQSSVSFRSGGSRSFSTASAITPSVSRTSFTSVSRSGGGGGGGFGRVSLAGACGVGGYGSRSLYNLGGSKRISISTSGGSFRNRFGAGAGGGYGFGGGAGSGFGFGGGAGGGFGLGGGAGFGGGFGGPGFPVCPPGGIQEVTVNQSLLTPLNLQIDPSIQRVRTEEREQIKTLNNKFASFIDKVRFLEQQNKVLDTKWTLLQEQGTKTVRQNLEPLFEQYINNLRRQLDSIVGERGRLDSELRNMQDLVEDFKNKYEDEINKRTTAENEFVMLKKDVDAAYMNKVELEAKVDALMDEINFMKMFFDAELSQMQTHVSDTSVVLSMDNNRNLDLDSIIAEVKAQYEEIANRSRTEAESWYQTKYEELQQTAGRHGDDLRNTKHEISEMNRMIQRLRAEIDNVKKQCANLQNAIADAEQRGELALKDARNKLAELEEALQKAKQDMARLLREYQELMNTKLALDVEIATYRKLLEGEECRLSGEGVGPVNISVVTSSVSSGYGSGSGYGGGLGGGLGGGLGGGLAGGSSGSYYSSSSGGVGLGGGLSVGGSGFSASSGRGLGVGFGSGGGSSSSVKFVSTTSSSRKSFKS

SEQ ID NO: 4 - protein, 472 aa, synthetic construct
MTTCSRQFTSSSSMKGSCGIGGGIGGGSSRISSVLAGGSCRAPSTYGGGLSVSSSRFSSGGACGLGGGYGGGFSSSSSSFGSGFGGGYGGGLGAGLGGGFGGGFAGGDGLLVGSEKVTMQNLNDRLASYLDKVRALEEANADLEVKIRDWYQRQRPAEIKDYSPYFKTIEDLRNKILTATVDNANVLLQIDNARLAADDFRTKYETELNLRMSVEADINGLRRVLDELTLARADLEMQIESLKEELAYLKKNHEEEMNALRGQVGGDVNVEMDAAPGVDLSRILNEMRDQYEKMAEKNRKDAEEWFFTKTEELNREVATNSELVQSGKSEISELRRTMQNLEIELQSQLSMKASLENSLEETKGRYCMQLAQIQEMIGSVEEQLAQLRCEMEQQNQEYKILLDVKTRLEQEIATYRRLLEGEDAHLSSSQFSSGSQSSRDVTSSSRQIRTKVMDVHDGKVVSTHEQVLRTKN

SEQ ID NO: 5 - protein, 564 aa, synthetic construct
MASTSTTIRSHSSSRRGFSANSARLPGVSRSGFSSVSVSRSRGSGGLGGACGGAGFGSRSLYGLGGSKRISIGGGSCAISGGYGSRAGGSYGFGGAGSGFGFGGGAGIGFGLGGGAGLAGGFGGPGFPVCPPGGIQEVTVNQSLLTPLNLQIDPTIQRVRAEEREQIKTLNNKFASFIDKVRFLEQQNKVLETKWTLLQEQGTKTVRQNLEPLFEQYINNLRRQLDSIVGERGRLDSELRGMQDLVEDFKNKYEDEINKRTAAENEFVTLKKDVDAAYMNKVELQAKADTLTDEINFLRALYDAELSQMQTHISDTSVVLSMDNNRNLDLDSIIAEVKAQYEEIAQRSRAEAESWYQTKYEELQVTAGRHGDDLRNTKQEIAEINRMIQRLRSEIDHVKKQCANLQAAIADAEQRGEMALKDAKNKLEGLEDALQKAKQDLARLLKEYQELMNVKLALDVEIATYRKLLEGEECRLNGEGVGQVNISVVQSTVSSGYGGASGVGSGLGLGGGSSYSYGSGLGVGGGFSSSSGRAIGGGLSSVGGGSSTIKYTTTSSSSRKSYKH

SEQ ID NO: 6 - protein, 564 aa, synthetic construct
MASTSTTIRSHSSSRRGFSANSARLPGVSRSGFSSISVSRSRGSGGLGGACGGAGFGSRSLYGLGGSKRISIGGGSCAISGGYGSRAGGSYGFGGAGSGFGFGGGAGIGFGLGGGAGLAGGFGGPGFPVCPPGGIQEVTVNQSLLTPLNLQIDPAIQRVRAEEREQIKTLNNKFASFIDKVRFLEQQNKVLDTKWTLLQEQGTKTVRQNLEPLFEQYINNLRRQLDNIVGERGRLDSELRNMQDLVEDLKNKYEDEINKRTAAENEFVTLKKDVDAAYMNKVELQAKADTLTDEINFLRALYDAELSQMQTHISDTSVVLSMDNNRNLDLDSIIAEVKAQYEEIAQRSRAEAESWYQTKYEELQITAGRHGDDLRNTKQEIAEINRMIQRLRSEIDHVKKQCANLQAAIADAEQRGEMALKDAKNKLEGLEDALQKAKQDLARLLKEYQELMNVKLALDVEIATYRKLLEGEECRLNGEGVGQVNISVVQSTVSSGYGGASGVGSGLGLGGGSSYSYGSGLGVGGGFSSSSGRATGGGLSSVGGGSSTIKYTTTSSSSRKSYKH

SEQ ID NO: 7 - protein, 473 aa, synthetic construct
MTTCSRQFTSSSSMKGSCGIGGGIGGGSSRISSVLAGGSCRAPSTYGGGLSVSSRFSSGGACGLGGGYGGGFSSSSSFGSGFGGGYGGGLGAGFGGGLGAGFGGGFAGGDGLLVGSEKVTMQNLNDRLASYLDKVRALEEANADLEVKIRDWYQRQRPSEIKDYSPYFKTIEDLRNKIIAATIENAQPILQIDNARLAADDFRTKYEHELALRQTVEADVNGLRRVLDELTLARTDLEMQIEGLKEELAYLRKNHEEEMLALRGQTGGDVNVEMDAAPGVDLSRILNEMRDQYEQMAEKNRRDAETWFLSKTEELNKEVASNSELVQSSRSEVTELRRVLQGLEIELQSQLSMKASLENSLEETKGRYCMQLSQIQGLIGSVEEQLAQLRCEMEQQSQEYQILLDVKTRLEQEIATYRRLLEGEDAHLSSQQASGQSYSSREVFTSSSSSSSRQTRPILKEQSSSSFSQGQSS

SEQ ID NO: 8 - protein, 432 aa, synthetic construct
MTTSIRQFTSSSSIKGSSGLGGGSSRTSCRLSGGLGAGSCRLGSAGGLGSTLGGSSYSSCYSFGSGGGYGSSFGGVDGLLAGGEKATMQNLNDRLASYLDKVRALEEANTELEVKIRDWYQRQAPGPARDYSQYYRTIEELQNKILTATVDNANILLQIDNARLAADDFRTKFETEQALRLSVEADINGLRRVLDELTLARADLEMQIENLKEELAYLKKNHEEEMNALRGQVGGEINVEMDAAPGVDLSRILNEMRDQYEKMAEKNRKDAEDWFFSKTEELNREVATNSELVQSGKSEISELRRTMQALEIELQSQLSMKASLEGNLAETENRYCVQLSQIQGLIGSVEEQLAQLRCEMEQQNQEYKILLDVKTRLEQEIATYRRLLEGEDAHLTQYKKEPVTTRQVRTIVEEVQDGKVISSREQVHQTTR